(19) United States
(12) Patent Application Publication
(10) Pub. No.: US 2024/0002479 A1
(43) Pub. Date: Jan. 4, 2024

(54) SINGLE-CHAIN ANTIBODY AGAINST FLAVIVIRUS NS1 PROTEIN

(71) Applicants: THE REGENTS OF THE UNIVERSITY OF MICHIGAN, Ann Arbor, MI (US); THE REGENTS OF THE UNIVERSITY OF CALIFORNIA, Oakland, CA (US)

(72) Inventors: Janet L. Smith, Ann Arbor, MI (US); David L. Akey, Ann Arbor, MI (US); William Clay Brown, Ann Arbor, MI (US); Eva Harris, Oakland, CA (US); Scott B. Biering, Oakland, CA (US); P. Robert Beatty, Oakland, CA (US)

(21) Appl. No.: 18/253,886

(22) PCT Filed: Nov. 23, 2021
(86) PCT No.: PCT/US21/60468

§ 371 Date: May 22, 2023

(51) Int. Cl. C07K 16/10; A61P 37/04; C12N 15/63

(52) U.S. Cl. CPC C07K 16/1081; A61P 37/04; C07K 2317/622; C07K 2317/565; C07K 2317/24; C12N 15/63

(57) ABSTRACT

The disclosure is directed to binding agents, e.g., a single chain variable fragment (scFv) that specifically binds to a flavivirus NS1 protein, as well as compositions comprising the binding agent and a method of using such compositions to induce an immune response against a flavivirus (e.g., a dengue virus). The disclosure also provides a conjugate comprising the binding agent, and a recombinant NS1 antigen.

### CROSS-REFERENCE TO RELATED APPLICATIONS

This application claims the benefit of U.S. Provisional Application No. 63/117,222 filed Nov. 23, 2020, the contents of which is herein incorporated by reference in their entirety.

### STATEMENT REGARDING FEDERAL FUNDING

This invention was made with government support under AI124493 and AI130130 awarded by the National Institutes of Health. The government has certain rights in the invention.

### SEQUENCE LISTING

The text of the computer readable sequence listing filed herewith, titled “38953-601_SEQUENCE_LISTING_ST25”, created Nov. 23, 2021, having a file size of 62,755 bytes, is hereby incorporated by reference in its entirety.

### FIELD

The disclosure provides antigen-binding agents that specifically bind to flavivirus NS1 protein.

### BACKGROUND

Flaviviruses are emerging arthropod-borne viruses representing an immense global health problem. The prominent viruses of this group include dengue virus, yellow fever virus, Japanese encephalitis virus, West Nile virus, tick borne encephalitis virus, and Zika Virus. Flaviviruses are endemic in many parts of the world and are responsible for illnesses ranging from mild flu like symptoms to severe hemorrhagic, neurologic, and cognitive manifestations leading to death. Flaviviruses have the potential to emerge and outbreak in non-endemic geographical regions, but the development of vaccines has been challenging. There are currently no approved anti-flaviviral therapeutics available.

Dengue virus serotypes 1-4 (DENV1-4) are mosquito-borne flaviviruses causing 50-100 million disease cases and about 500,000 hospitalizations annually, with severe forms of disease manifesting in vascular leak as a result of endothelial dysfunction (1, 2). The trigger(s) of these pathologies are often broadly described as a “cytokine storm” resulting from uncontrolled viral replication and activation of target immune cells, with a direct pathogenic role now characterized for the DENV non-structural protein 1 (NS1) via interactions with endothelial and immune cells (3-5). There are currently no approved therapeutics for dengue, and the only licensed DENV vaccine, DENGVAXIA®, is now reserved strictly for patients with preexisting DENV immunity due to the risk of predisposing DENV-naïve patients to severe dengue disease, presumably via antibody-dependent enhancement (ADE) (6, 7). This risk has made a successful vaccine targeting the DENV envelope protein (E) challenging.

There remains a need for compositions and methods for treating and preventing flavivirus infections.

### BRIEF SUMMARY OF THE INVENTION

The disclosure provides an agent which binds to a flavivirus NS1 protein and comprises three complementarity determining regions (CDRs) of an antibody heavy chain variable region (VH) and three CDRs of an antibody light chain variable region, wherein: (a) CDR1 of the VH (HCDR1) comprises the amino acid sequence of SEQ ID NO: 1, CDR2 of the VH (HCDR2) comprises the amino acid sequence of SEQ ID NO: 2, and CDR3 of the VH (HCDR3) comprises the amino acid sequence of SEQ ID NO: 3; and (b) CDR1 of the LH (LCDR1) comprises the amino acid sequence of SEQ ID NO: 4, CDR2 of the LH (LCDR2) comprises the amino acid sequence of SEQ ID NO: 5, and CDR3 of the LH (LCDR3) comprises the amino acid sequence of SEQ ID NO: 6.

The disclosure also provides a binding agent that specifically binds to a region of a flavivirus NS1 protein comprising one or more of the following amino acid residues: (a) H269, L270, E281, G282, R299, T301, V303, T304, G305, T307, E326, D327, G328, W330, E343, N344, L345, V346, K347, S348, and/or M349 of Dengue virus 1 (NCBI Accession No. P17763.2); (b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990); (c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843); (d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2); (e) H269, S270, P281, G282, R299, T301, A303, S304, G305, V307, K326, D327, G328, W330, S343, N344, L345, V346, R347, S348, and/or M349 of Zika virus (NCBI Accession No. AZS35340); (f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2); (g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4); (h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395); (i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135); (j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1); (k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492); (l) D271, Q272, P283, G284, R301, T303, E305, S306, G307, I309, G328, T329, D330, W332, G344, G345, L346, V347, R348, S349, and/or M350 of Powassan virus (NCBI Accession No. ACD88752); and/or (m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

The disclosure further provides a composition comprising a recombinant NS1 antigen and a pharmaceutically acceptable carrier, which recombinant NS1 antigen comprises one or more of the following amino acid residues: (a) H269, L270, E281, G282, R299, T301, V303, T304, G305, T307, E326, D327, G328, W330, E343, N344, L345, V346, K347, S348, and/or M349 of Dengue virus 1 (NCBI Accession No. P17763.2); (b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990); (c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843); (d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2); (e) H269, S270, P281, G282, R299, T301, A303, S304, G305, V307, K326, D327, G328, W330, 5343, N344, L345, V346, R347, S348, and/or M349 of Zika virus (NCBI Accession No. AZS35340); (f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2); (g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4); (h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395); (i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135); (j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1); (k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492); (l) D271, Q272, P283, G284, R301, T303, E305, S306, G307, T309, G328, T329, D330, W332, G344, G345, L346, V347, R348, S349, and/or M350 of Powassan virus (NCBI Accession No. ACD88752); or (m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

Also provided is a composition comprising a nucleic acid sequence encoding a recombinant flavivirus NS1 antigen and pharmaceutically acceptable carrier, wherein the recombinant NS1 antigen comprises (a) H269, L270, E281, G282, R299, T301, V303, T304, G305, T307, E326, D327, G328, W330, E343, N344, L345, V346, K347, S348, and/or M349 of Dengue virus 1 (NCBI Accession No. P17763.2); (b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990); (c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843); (d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2); (e) H269, S270, P281, G282, R299, T301, A303, S304, G305, V307, K326, D327, G328, W330, S343, N344, L345, V346, R347, S348, and/or M349 of Zika virus (NCBI Accession No. AZS35340); (f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2); (g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4); (h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395); (i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135); (j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1); (k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492); (l) D271, Q272, P283, G284, R301, T303, E305, S306, G307, T309, G328, T329, D330, W332, G344, G345, L346, V347, R348, S349, and/or M350 of Powassan virus (NCBI Accession No. ACD88752); or (m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

The disclosure provides methods of inducing an immune response against flaviviruses in a mammal using the aforementioned binding agents and compositions comprising same.

### BRIEF DESCRIPTION OF THE DRAWING(S)

FIG. 1A is a survival curve of Ifnar^{-/-} mice infected with DENV2-D220. Mice were given two 150-μg doses (300 μg for “2B7 high”) of full-length 2B7, a 2B7 single-chain variable fragment (scFv), an anti-E antibody (4G2), or an isotype control antibody the day before and after infection. Numbers in parentheses indicate the number of mice in each group. FIG. 1B is an image showing localized leak of the tracer molecule, dextran-647, measured after dorsal intradermal injection of NS1 with or without 2B7, or the indicated controls, into the shaved backs of mice. One representative experiment of n=6 mice is displayed. FIG. 1C is a graph showing quantification of the data shown in FIG. 1B as mean fluorescence intensity (MFI). FIG. 1D is a graph illustrating results of a TEER assay of HPMEC hyperpermeability after addition of DENV2 NS1 with or without 2B7, or the indicated controls, at the indicated time-points post-NS1 treatment (n=3 biological replicates). FIG. 1E is a series of images showing endothelial dysfunction and EGL disruption that were monitored using immunofluorescent microscopy 6 hours post-treatment of DENV2 NS1 with or without 2B7, 2B7 Fab, or the indicated controls. Endothelial cell dysfunction (bottom, n=2 biological replicates) was monitored using the cathepsin L-activity reporter molecule, Magic Red, and EGL disruption (top, n=4 biological replicates) was monitored by staining sialic acid on the cell surface. FIGS. 1F and 1G are graphs showing quantification of the data presented in FIG. 1E. FIG. 1H is a series of images showing DENV2 NS1 binding to HPMEC in the presence of 2B7, 2B7 Fab, or the indicated controls, which was monitored by immunofluorescent microscopy 90 minutes post-NS1 treatment (n=3 biological replicates). FIG. 1I is a graph showing quantification of the data presented in FIG. 1H. For all figures, scale bars are 50 μm. n.s., not significant p>0.05; *p<0.05; **p<0.01; ***p<0.001; ****p<0.0001 by one-way ANOVA analysis with multiple comparisons.

FIG. 2A is a schematic diagram showing perpendicular views of a 3.3-Å crystal structure of 2B7 Fab (heavy chain, dark blue; light chain, light blue) and DENV1 NS1 dimer (β-ladder domains, green; β-roll and wing domains, cyan). The combining site is boxed (yellow) in the lower image, right monomer. FIG. 2B is a schematic diagram showing the DENV2 NS1 epitope for 2B7 scFv (colored by conservation across flaviviruses according to the key and based on the alignment in FIG. 3A), with surfaces outside the epitope in gray. Sites of mutagenesis in (see FIG. 3) are labeled. FIG. 2C is a schematic diagram showing 2B7 scFv complementarity-determining regions (CDRs, in tube rendering) for the heavy chain (dark blue) and light chain (light blue) overlaid on the DENV2 NS1 epitope surface. Surfaces of amino acids conserved among the four DENV serotypes but variable in other flaviviruses are purple, other epitope residues are in green; view as in FIG. 2B. FIG. 2D is a schematic diagram showing detail of the 2B7 scFv and the DENV2 NS1 combining site highlighting the interacting amino acids. The 2B7 backbone is in blue, and NS1 is in green with key side chains shown as sticks. Pan-flavivirus conserved side chains (orange) are at the center of the discontinuous epitope; DENV-conserved side chains (yellow) are at the epitope periphery: hydrogen bonds are shown as dashed lines.

FIG. 3A is an alignment of amino acids across the discontinuous 2B7 combining site in NS1 from DENV1-4, ZIKV, SLEV, WNV, JEV, USUV, WSLV, TBEV, POWV, and YFV (amino acids as indicated from SEQ ID NOs: 67-79, respectively). Residues in contact with 2B7 are boxed. FIG. 3B is a graph showing results of ELISAs measuring the interaction of 2B7 with NS1 from DENV1-4. Data displayed are at least n=3 biological replicates. FIG. 3C is a graph showing results of ELISAs measuring 2B7 interaction with other flavivirus NS1 proteins (at least n=3 biological replicates). FIGS. 3D and 3E are graphs showing data from ELISAs similar to data in FIG. 3C, except for the indicated DENV2 NS1 mutagenized proteins compared to the in-house produced (NS1-WT) or commercially purchased (NS1-WT-C) control proteins. Data displayed are n=3 biological replicates. FIG. 3F is a graph showing data from ELISAs similar to data in FIG. 3C, except for the indicated WNV or ZIKV NS1 mutagenized proteins (n=3 biological replicates). FIG. 3G is a graph showing WNV or ZIKV NS1 binding to HBMEC in the presence or absence of 25 μg/ml 2B7, and the indicated controls, as measured by immunofluorescent microscopy 90 minutes post-NS1 treatment. Data displayed are at least n=4 biological replicates. FIG. 3H is a graph showing the same data presented in FIG. 3G but with the indicated concentrations of 2B7 (at least n=3 biological replicates). FIG. 3I is a graph showing the effect of WNV or ZIKV NS1 on hyperpermeability of HBMEC as measured using TEER, in the presence or absence of 25 μg/ml 2B7, or the indicated controls. AUC is the negative area under the curve (see FIG. 12C) correlating with a drop in endothelial cell monolayer electrical resistance. Data presented are n=2 biological replicates. FIG. 3J is a graph showing the same data as presented in FIG. 3I, but with the indicated concentration of 2B7 (at least n=3 biological replicates). **p<0.01; ***p<0.001; ****p<0.0001 by one-way ANOVA analysis with multiple comparisons. FV, flavivirus.

FIG. 4A is a schematic diagram showing 2B7 Fab (blue ribbon) in complex with DENV1 NS1 hexamer above a schematic of the plasma membrane, illustrating 2B7 Fab-mediated steric hinderance of NS1 membrane interaction. The NS1 surface is colored by dimer (light/dark green at right, gray at left and tan in the back), with hydrophobic regions in yellow for all three dimers. The 2B7 Fab is bound to both subunits of the green dimer and occludes cell surface interaction of the yellow wing-domain hydrophobic loop (centered on the WWG motif). FIG. 4B is a schematic diagram showing perpendicular views of 2B7 Fab complex with DENV1 NS1 dimer, illustrating Fab interference with membrane interaction of the NS1 hydrophobic face (yellow). The left image shows the dimer hydrophobic face with wing hydrophobic loops at the periphery and central hydrophobic surface of the β-roll domain. This face is inside the hexamer and invisible in FIG. 4A. The NS1 epitope for 2B7 is in orange. FIG. 4C includes graphs showing endothelial hyperpermeability of HPMEC as monitored via TEER for the indicated DENV2 NS1 mutants compared to the commercial NS1-WT (NS1-WT-C) or in-house produced NS1-WT (NS1-WT). Data presented are n=2 biological replicates. FIG. 4D includes graphs showing cell binding to HPMEC of NS1 mutants or controls as monitored by immunofluorescent microscopy 90 minutes post-NS1 treatment (n=3 biological replicates). FIG. 4E is a series of images showing endothelial dysfunction as monitored using immunofluorescent microscopy and the cathepsin L-activity reporter molecule, Magic Red, 6 hours post-treatment of DENV2 NS1 mutants, or the indicated controls. Data presented are n=2 biological replicates. FIG. 4F is a graph showing quantification of the data presented in FIG. 4E. n.s., not significant p>0.05; *p<0.05; **p<0.01 by one-way ANOVA analysis with multiple comparisons.

FIGS. 5A-5C show that anti-NS1 mAb 2B7 is protective against dengue virus pathogenesis and NS1-mediated vascular leak in vivo (related to FIG. 1). FIG. 5A includes graphs of morbidity scores of Ifnar^{-/-} mice infected with 5×10⁵ PFU of DENV2-D220 from FIG. 1A. Morbidity increases with score number. Mice assigned a score of 1 were healthy, while those with a score of 2 displayed mild signs of lethargy with some fur ruffling, but no hunching. Mice assigned a score of 3 showed fur ruffling, were hunched, and showed mild signs of lethargy. Mice given a score of 4 were ruffled, hunched, lethargic, and displayed limited mobility. Finally, mice receiving a score of 5 were moribund, the endpoint of the experiment, at which point they were euthanized. FIG. 5B is an image showing IgG control for the dorsal intradermal injection leak model shown in FIG. 1B. Shaved mouse backs were injected intradermally with the indicated treatments followed by an intravenous injection of dextran-647. Mice were sacrificed, and mouse backs were collected and analyzed by an Odyssey Licor fluorescent scanner. A representative experiment is shown from n=10 mice. FIG. 5C is a graph showing quantification of the data presented in FIG. 5B. Mean fluorescence intensity (MFI) is plotted±SEM, n.s., not significant p>0.05; *p<0.05; ***p<0.001.

FIGS. 6A-6E show that 2B7 inhibits NS1 cell binding and binds to the NS1 β-ladder (related to FIG. 1). FIG. 6A is graph showing results of an NS1 cell binding assay using 293F suspension cells. DENV2 NS1 was mixed with the indicated concentration of antibody and added to a U-bottom 96-well plate containing 293F cells. NS1 binding was assessed by flow cytometry. Data are presented as mean±SEM of n=3 biological replicates. FIG. 6B is a series of images showing DENV2 NS1 binding to HPMEC in the presence or absence of 15 μg/ml full-length 2B7, the 2B7 scFv, or the indicated controls, as measured by immunofluorescent microscopy 90 minutes post-NS1 treatment. Data presented are representative images from n=2 biological replicates. The scale bar is 50 μm. FIG. 6C is a graph showing quantification of the data presented in FIG. 6B presented as mean±SEM. FIG. 6D is a graph showing results of a direct ELISA using the indicated recombinant NS1 domains to coat plates. Proteins were detected with the indicated antibodies or sera, and absorbance was measured at 405 nm. Data are presented as mean±SEM of n=2 biological replicates. FIG. 6E is a series of graphs which show biolayer interferometry measurements of 2B7 Fab binding to immobilized full-length NS1 and the indicated NS1 domains. Full-length NS1 or the indicated domains were immobilized on anti-His tips. Tips were serially dipped into wells containing either increasing amounts of the 2B7 Fab fragment or buffer control (association step, rising signal) alternately with immersion into buffer only (disassociation step, falling signal). Results from n=2 biological replicates (NS1 and 2B7 Fab preparations) indicated K_{d} values in the range of 5-8 nM. Similar values were obtained for the full-length 2B7 and the scFv fragment.

FIGS. 7A and 7B show that phage display reveals that 2B7 binds to the β-ladder of NS1 (related to FIG. 2). FIG. 7A is an alignment of DENV1 and DENV2 NS1 amino acid sequences (SEQ ID NOs: 67 and 68, respectively) highlighting the two overlapping peptides within the β-ladder that were bound by 2B7 in the VirScan phage display system (peptide 1: amino acids 260-316; peptide 2: amino acids 288-344), with the discontinuous 2B7 epitope highlighted in magenta. FIG. 7B is an NS1 dimer surface rendering with the 2B7 Fab bound to the bright green subunit. Data in FIGS. 7A and 7B are graphical representations from n=2 biological replicates.

FIGS. 8A and 8B are schematic diagrams showing the structure of the 2B7-NS1 complex highlighting NS1 domains (related to FIG. 2). Perpendicular views of DENV1 NS1:2B7 Fab (FIG. 8A) and DENV2 NS1:2B7 scFv (FIG. 8B) complexes are shown. NS1 dimer surface is colored by domain (blue β-roll, cyan wing, green β-ladder; darker shade in one subunit, lighter in the other) with bound 2B7 Fab/scFv in ribbon rendering (dark blue heavy chain, light blue light chain). The lower panel in FIG. 8A and FIG. 8B is the NS1 surface that faces the inside of the hexamer. The domains are labeled for the darker-shaded subunit.

FIGS. 9A-9E are schematic diagrams showing that 2B7 binds identically to DENV1 and DENV2 NS1 (related to FIG. 2). FIG. 9A shows the combination of site detail with electron density at 2.89 Å (2Fo-Fc contoured at 1 σ) for DENV2 NS1 with the 2B7 scFv, and FIG. 9B shows the combination of site detail with electron density at 3.3 Å (2Fo-Fc contoured at 1 σ) for DENV1 NS1 complexed with the 2B7 Fab. FIG. 9C shows electron density at 4.2 Å (2Fo-Fc contoured at 1 σ) of the DENV2 NS1 dimer with 2B7 Fab, shown in the same orientations as in FIG. 2A. Well-defined density is present for all domains of NS1 and the 2B7 Fab. FIG. 9D is a ribbon diagram of the DENV2 NS1 dimer (light and dark green subunits) with the 2B7 Fab (dark blue heavy chain, light blue light chain), as in FIG. 9C with the density removed. FIG. 9E shows superposition of DENV1 (gray) and DENV2 NS1 (colored as in FIG. 9D) dimers in complex with the 2B7 Fab, viewed as in FIG. 9D. The superposition was based on the NS1 dimer and shows identical 2B7 Fab binding to DENV1 and DENV2 NS1. FIG. 9F is a table showing NS1 epitope residues and corresponding contacts in 2B7.

FIGS. 10A and 10B show selection and production of DENV2 NS1 mutants (related to FIG. 3). FIG. 10A is a series of graphs showing results of capture ELISA with supernatants from DENV2 NS1 mutant-transfected 293T cells. Plates were coated with mAb (7E11) targeting the NS1 wing domain, and captured protein was detected using either 2B7 or an anti-HIS antibody. Detected proteins were measured via absorbance at 450 nm. Displayed is a single representative experiment from n=3 biological replicates. FIG. 10B is a series of images Western blots of the same samples tested in FIG. 10A using the anti-NS1 wing mAb 7E11. Displayed is one representative experiment from n=3. In all panels, NS1 proteins selected for further analysis are indicated with stars. FV, flavivirus.

FIGS. 11A-11C show production and quality control of WNV and ZIKV NS1 mutants (related to FIG. 3). FIG. 11A is an image of Western blot analysis (anti-NS1 antibody, “7E11”) following SDS-PAGE of WT and mutant WNV/ZIKV NS1 proteins after purification. FIG. 11B is an image of silver stain following SDS-PAGE of purified WT and mutant WNV/ZIKV NS1 proteins. FIG. 11C is an image of Western blot analysis (anti-NS1 antibody, “7E11”) following native-PAGE of purified WT and mutant WNV/ZIKV NS1 proteins.

FIGS. 12A-12C show that 2B7 inhibits Zika virus and West Nile virus NS1 cell binding and NS1-mediated endothelial hyperpermeability (related to FIG. 3). FIG. 12A is an image showing WNV or ZIKV NS1 (10 μg/mL) binding to HBMEC in the presence or absence of 2B7, alongside the indicated controls, as measured by immunofluorescent microscopy 90 minutes post-NS1 treatment. Panels are representative images from FIG. 3G from n=4 biological replicates. The scale bar is 50 μm. FIG. 12B includes images showing WNV or ZIKV NS1 (10 μg/mL) binding to HBMEC in the presence or absence of 2B7, alongside the indicated controls, as measured by immunofluorescent microscopy 90 minutes post-NS1 treatment. Panels are representative images from FIG. 3H from at least n=3 biological replicates. FIG. 12C includes a series of graphs showing results of a TEER assay used to measure the capacity of 2B7 to inhibit flavivirus NS1-mediated endothelial hyperpermeability of HBMEC and HPMEC. 5 μg/mL of the indicated NS1 protein was preincubated with the indicated concentration of 2B7 for 30 minutes at 37° C. before being added to the apical chamber of transwells. Data are the full curves plotted from FIGS. 3I and 3J and represent n=2 biological replicates. UT, untreated.

FIGS. 13A-13D are graphs demonstrating that 2B7 does not mediate antibody-dependent enhancement of flavivirus infection (related to FIG. 3). FIGS. 13A and 13B show results of virus neutralization assays where flavivirus-permissive human U937-DC-SIGN monocytic cells were infected with DENV (MOI=0.01), ZIKV (MOI=0.01), or KUNV (MOI=0.1) in the presence of the indicated dose of an anti-envelope mAb 4G2 (FIG. 13A) or the anti-NS1 mAb 2B7 (FIG. 13B). FIGS. 13C and 13D show results of ADE assays using non-flavivirus-permissive K562 cells infected with DENV, ZIKV, or KUNV in the presence of the indicated dose of mAb 4G2 (FIG. 13C) or mAb 2B7 (FIG. 13D). mAb dilution range is 5,000-2.3 ng/mL. Data are from n=2 biological replicates. MOT, multiplicity of infection.

FIGS. 14A-14C show that 2B7 does not modulate the clotting cascade or bind to endothelial cells (related to FIG. 3). FIG. 14A is a graph showing results of an in vitro anticoagulant activity assay. 2B7 or an anti-fibrinogen antibody were incubated with human plasma for 10 minutes to initiate plasma clotting followed by an activated partial thromboplastin time (APPT) assay. Clotting time is displayed in seconds. PBS and heparin (0.1 ng/ml) were used as negative and positive controls, respectively. NC, no coagulation. Data plotted are mean f SEM. FIG. 14B is a series of images showing results of an antibody cell binding assay using HPMEC and HBMEC treated or not with 10 μg/ml of NS1 for 1 hour followed by treatment or not with 25 μg/ml 2B7 or an IgG control. Binding of antibody was measured by immunofluorescent microscopy 60 minutes posttreatment. Panels are representative images of n=3 (HPMEC) and n=2 (HBMEC) biological replicates. The scale bar is 50 μm. FIG. 14C is a graph showing quantification of the data in FIG. 14B presented as mean±SEM. n.s., not significant p>0.05; ****p<0.0001.

FIG. 15 is an alignment of the NS1 wing flexible loop from DENV1-4, ZIKV, SLEV, WNV, JEV, USUV, WSLV, TBEV, POWV, and YFV (amino acids as indicated from SEQ ID NOs: 67-79, respectively) NS1 proteins demonstrating high levels of conservation for residues W115, W118, and G119.

FIGS. 16A-F show production and quality control of DENV2 NS1 mutants (related to FIG. 4). FIG. 16A is an image of western blot analysis (anti-HIS antibody) following SDS-PAGE of WT and mutant NS1 proteins after purification. FIG. 16B is an image of silver stain following SDS-PAGE of purified WT and mutant NS1 proteins. FIG. 16C includes images from western blot analysis (anti-His antibody) following native-PAGE of purified WT and mutant NS1 proteins. FIG. 16D is a graph showing results of size-exclusion chromatography of purified WT and mutant NS1 proteins. FIG. 16E is a series of images showing results from western blot analysis (anti-His antibody) following SDS-PAGE of the indicated size-exclusion chromatography fractions from FIG. 16D. FIG. 16F includes images from western blot analysis (anti-HIS antibody) following SDS-PAGE of WT and mutant NS1 proteins after incubation in endothelial cell medium at 37° C. and 5% CO₂ for the indicated times. FIG. 16G is a graph showing results of direct ELISAs of purified WT and mutant DENV2 NS1 proteins with wing domain substitutions, detected by 2B7. NS1-WT-C is the commercially purchased WT NS1, while NS1-WT is the in-house produced WT NS1. Unless otherwise indicated, the data presented are from n=3 biological replicates.

FIGS. 17A-17C show results illustrating that a DENV2 NS1 wing domain mutant is defective for cell binding, while DENV2 NS1 O-ladder mutants are defective for downstream pathogenesis (related to FIG. 4). FIG. 17A includes graphs showing results of a TEER assay used to measure the capacity of mutant DENV2 NS1 proteins to mediate endothelial hyperpermeability of HPMEC. Five μg/mL of the indicated NS1 proteins were added to the apical chamber of transwells. Data are the full curves plotted from FIG. 4C. Data are presented as mean t SEM of n=2 biological replicates for the left panel and one representative experiment for n=2 biological replicates for right panel. FIG. 17B is a series of graphs illustrating NS1 binding to 293F suspension cells. WT or mutant NS1 proteins and the indicated concentration of mAb 2B7 were added to a U-bottom 96-well plate, and NS1 binding was assessed by flow cytometry using an anti-His antibody conjugated to Alexa Fluor 647. Substitutions in the NS1 β-ladder (top and middle panels) and the wing domain (bottom panel) were compared to NS1-WT controls. Data are presented as mean±SEM of n=3 biological replicates, except for the middle plot which is a single representative experiment from n=3 biological replicates. FIG. 17C is a series of images showing results of an NS1 cell binding assay using HPMECs. Ten μg/ml of the indicated NS1 proteins were added to cells seeded on coverslips before live-staining with anti-His antibody conjugated to Alexa Fluor 647, fixation in 4% formaldehyde, and imaging by immunofluorescent microscopy. The mean fluorescence intensity (MFI) of these images are quantified in FIG. 4D. Data are representative of n=3 biological replicates. NS1-WT-C is the commercially purchased WT NS1, while NS1-WT is the in-house produced WT NS1. For all figures, scale bars are 50 μm.

### DETAILED DESCRIPTION

The present disclosure is predicated, at least in part, on the identification of three crystal structures of full-length dengue virus (DENV) NS1 protein complexed with a flavivirus cross-reactive NS1-specific monoclonal antibody, 2B7, revealing a protective mechanism by which two domains of NS1 are antagonized simultaneously. A single chain variable fragment (scFv) derived from the 2B7 antibody has been generated that binds to the NS1 protein in the same manner as a 2B7 Fab and full-length 2B7 antibody. As described herein, the 2B7 scFv blocks NS1 protein binding to cell surfaces and the triggering of the endothelial dysfunction associated with the most severe forms of Flavivirus diseases. The present disclosure provides a mechanistic explanation for 2B7 protection against NS1-induced pathology and demonstrates that the 2B7 antibody, and fragments, derivatives, or analogs thereof, may be used to treat infections by multiple different flaviviruses. However, the invention is not limited to any particular mechanism of action and an understanding of the mechanism is not necessary to practice the invention.

### Definitions

To facilitate an understanding of the present technology, a number of terms and phrases are defined below. Additional definitions are set forth throughout the detailed description.

The terms “nucleic acid,” “polynucleotide,” “nucleotide sequence,” and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

The terms “peptide,” “polypeptide,” and “protein” are used interchangeably herein and refer to a polymeric form of amino acids of any length, which can include coded and non-coded amino acids, chemically or biochemically modified or derivatized amino acids, and polypeptides having modified peptide backbones.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In some embodiments, an epitope is a region of an antigen that is specifically bound by an antibody. In other embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. The antigen can be a protein or peptide of viral, bacterial, parasitic, fungal, protozoan, prion, cellular, or extracellular origin, which provokes an immune response in a mammal, preferably leading to protective immunity.

The term “binding agent,” as used herein, refers to a molecule, ideally a proteinaceous molecule, which specifically binds to another molecule, such as another protein. By “antigen-binding agent” is meant a molecule, such as a proteinaceous molecule, that specifically binds to an antigen. The antigen-binding agent comprises at least two components which, in combination, form the antigen-binding site of an antigen-binding agent. In some embodiments, a first component of an antigen-binding agent comprises an antibody heavy chain or a fragment thereof, and a second component of antigen-binding agent comprises an antibody light chain or fragment thereof.

The term “immunoglobulin” or “antibody,” as used herein, refers to a protein that is found in blood or other bodily fluids of vertebrates, which is used by the immune system to identify and neutralize foreign objects, such as bacteria and viruses. Typically, an immunoglobulin or antibody is a protein that comprises at least one complementarity determining region (CDR). The CDRs form the “hypervariable region” of an antibody, which is responsible for antigen binding (discussed further below). A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the CDRs. There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, N.Y. (2001)).

The framework regions are connected by three CDRs. As discussed above, the three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

The terms “fragment of an antibody,” “antibody fragment,” and “antigen-binding fragment” of an antibody are used interchangeably herein to refer to one or more fragments of an antibody that retain the ability to specifically bind to an antigen (see, generally, Holliger et al., Nat. Biotech., 23(9): 1126-1129 (2005)). Any antigen-binding fragment of the antibody described herein is within the scope of the invention. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and C_{H1} domains, (ii) a F(ab′)2 fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)2 fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

The term “monoclonal antibody,” as used herein, refers to an antibody produced by a single clone of B lymphocytes that is directed against a single epitope on an antigen. Monoclonal antibodies typically are produced using hybridoma technology, as first described in Köhler and Milstein, Eur. J. Immunol., 5: 511-519 (1976). Monoclonal antibodies may also be produced using recombinant DNA methods (see, e.g., U.S. Pat. No. 4,816,567), isolated from phage display antibody libraries (see, e.g., Clackson et al. Nature, 352: 624-628 (1991)); and Marks et al., J. Mol. Biol., 222: 581-597 (1991)), or produced from transgenic mice carrying a fully human immunoglobulin system (see, e.g., Lonberg, Nat. Biotechnol., 23(9): 1117-25 (2005), and Lonberg, Handb. Exp. Pharmacol., 181: 69-97 (2008)). In contrast, “polyclonal” antibodies are antibodies that are secreted by different B cell lineages within an animal. Polyclonal antibodies are a collection of immunoglobulin molecules that recognize multiple epitopes on the same antigen.

A “chimeric” antibody is an antibody or fragment thereof comprising both human and non-human regions (e.g., variable regions from a mouse antibody and constant regions from a human antibody). A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody.

The term “recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Alternatively, DNA sequences encoding RNA that is not translated may also be considered recombinant. Thus, the term “recombinant” nucleic acid also refers to a nucleic acid which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, the artificial combination may be performed to join nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention but may comprise a naturally occurring amino acid sequence.

A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

### Flavivirus NS1 Protein

The disclosure provides a binding agent that specifically binds to a flavivirus NS1 protein. Flaviviruses are enveloped, positive-sense, single-stranded RNA viruses. The RNA genome of the flaviviruses contains the 5′ cap (7mG) and 3′ CU—OH conserved tail, which directly translates into a long polypeptide in the cytoplasm of infected cells. The polypeptide is cleaved and processed by host and viral proteases into three structural proteins: envelope protein (E), capsid protein (C) and precursor membrane protein (prM), and seven non-structural components (NS1, NS2A, NS2B, NS3, NS4A, NS4B and NS5) (Rastogi et al., Virol J., 13: 131 (2016)). Among the non-structural proteins, NS1 is a highly conserved, dimer protein with the molecular weight ranges from 46-55 kDa depending on the extent of glycosylation. NS1 exists as a monomer, a dimer (membrane-bound protein, mNS1), and a hexamer (secreted protein, sNS1).

Extracellular NS1 acts as a virulence factor inhibiting complement, activating platelets and immune cells, and directly interacting with endothelial cells (11-13). This results in disruption of the endothelial glycocalyx layer (EGL) and intercellular junctional complexes, which are both critical for maintaining endothelial barrier integrity (13-15). NS1-mediated endothelial dysfunction is observed for multiple medically relevant mosquito-borne flaviviruses, including Zika (ZIKV), West Nile (WNV), Japanese encephalitis (JEV), and yellow fever (YFV) viruses (16, 17). With a prominent role in flavivirus pathogenesis, NS1 has emerged as a promising vaccine candidate. Indeed, vaccination with NS1 protects against lethal DENV or ZIKV challenge in mice (3, 18-20). Flavivirus NS1 has three domains that may possess distinct functions (21): a small “β-roll” dimerization domain (e.g., amino acids 1-29 of West Nile virus (WNV) and dengue virus type 2 (DENV2)), a “wing” domain protruding from the central β-domain like a wing (e.g., amino acids 30-180 of WNV and DENV2), and the “β-ladder domain” (e.g., amino acids 181-352 of WNV and DENV2), which is the predominant structural feature of NS1 (Akey et al., Science, 343: 881-885 (2014)). Despite a plethora of structural data (21-25), the mechanistic basis for antibody-mediated protection against NS1-induced endothelial dysfunction and the specific functional domains of NS1 responsible for different pathogenic functions are unknown. The binding agents described herein may bind to an NS1 protein of any flavivirus, of which there are over 50 known species. The majority of known members in the genus Flavivirus are arthropod borne (e.g., mosquito- or tick-borne), and many are important human and veterinary pathogens. Examples of mosquito-borne flaviviruses include yellow fever virus, dengue virus, Japanese encephalitis virus, West Nile virus, and Zika virus. Examples of tick-borne flaviviruses that cause encephalitis and hemorrhagic diseases include tick-borne encephalitis (TBE), Kyasanur Forest Disease (KFD) virus, Alkhurma disease virus, and Omsk hemorrhagic fever virus. Flavivirus classification and phylogeny is described in detail in, e.g., Schweitzer et al., Laboratory Medicine, 40(8): 493-499 (2009); DOI: 10.1309/LM5YWS85NJPCWESW; and Kuno et al., Journal of Virology, 72(1) 73-83 (1998); DOI: 10.1128/JVI.72.1.73-83.1998. In some embodiments, the binding agent specifically binds to an NS1 protein from yellow fever virus, dengue virus, Japanese encephalitis virus, West Nile virus, Zika virus, St. Louis encephalitis virus, tick-borne encephalitis virus, Usutu virus, Powassan virus, or Wesselsbron virus.

For example, the binding agent may specifically bind an NS1 protein from dengue virus. Dengue virus (DENV) is a mosquito-borne flavivirus that is estimated to cause up to 390 million infections, 96 million disease cases, and ˜500,000 hospitalizations annually (1). Infection with any of the four DENV serotypes (serotype 1 (DENV1), serotype 2 (DENV2), serotype 3 (DENV3), or serotype 4 (DENV4)) results in a range of syndromes from inapparent infection to classic dengue fever (DF) to dengue hemorrhagic fever/dengue shock syndrome (DHF/DSS), which is characterized by vascular leakage and shock (2). Most primary DENV infections caused by any of the four serotypes are asymptomatic or lead to the self-limiting but debilitating DF; however, secondary infections with a different (heterologous) DENV serotype can lead to increased risk of severe dengue (3). Immune responses after primary DENV infection lead to protective immunity to homologous secondary infection but may either protect against or cause increased disease severity in a subsequent DENV infection with a different serotype. The latter is thought to be mediated by serotype cross-reactive T cells or antibody-dependent enhancement (ADE), whereby cross-reactive antibodies that target viral structural proteins facilitate DENV infection of Fcγ receptor-bearing cells, leading to increased viral load (4, 5). ADE and cross-reactive T cells are thought to trigger an exaggerated and skewed immune response to a previously infecting serotype, resulting in a “cytokine storm,” i.e., rapid-onset, high-level production of proinflammatory cytokines, including tumor necrosis factor-α (TNF-α) and interleukin-6 (IL-6), in the blood that leads to endothelial permeability and vascular leak (6). However, the potential role of viral proteins in mediating vascular leakage has not been elucidated.

### Binding Agents

The binding agents provided herein desirably bind to a proteinaceous molecule, such as an antigen. In this case, therefore, a binding agent may be referred to as an “antigen-binding agent.” An antigen-binding agent may bind to a conformational epitope and/or a linear epitope present on a target antigen. The term “conformational epitope,” as used herein, refers to an antigenic protein composed of amino acid residues that are spatially near each other on the antigen's surface and are brought together by protein folding. In contrast, a “linear epitope” (also referred to as a “sequential epitope”) comprises a sequence of continuous amino acids that is sufficient for antibody binding. The binding agents described herein desirably specifically bind to particular amino acid residues of an NS1 protein from any suitable flavivirus. For example, the binding agent may bind to any one or combination of the amino acid residues from the flavivirus species set forth in Table 1.

TABLE 1

| | Genome NCBI | |
|---|---|---|
| Flavivirus | Accession No. | NS1 Amino acids |
| Dengue virus 1 | P17763.2 | H269, L270, E281, G282, R299, T301, V303, |
| | | T304, G305, T307, E326, D327, G328, W330, |
| | | E343, N344, L345, V346, K347, S348, M349 |
| Dengue virus 2 | P29990 | H269, L270, D281, G282, R299, T301, A303, |
| | | S304, G305, L307, E326, D327, G328, W330, |
| | | E343, N344, L345, V346, N347, S348, L349 |
| Dengue virus 3 | YP_001621843 | H269, L270, E281, G282, R299, T301, V303, |
| | | S304, G305, L307, E326, D327, G328, W330, |
| | | E343, N344, M345, V346, K347, S348, L349 |
| Dengue virus 4 | P09866.2 | H269, L270, P281, G282, R299, T301, A303, |
| | | S304, G305, L307, E326, D327, G328, W330, |
| | | E343, N344, M345, V346, K347, S348, Q349 |
| Zika virus | AZS35340 | H269, S270, P281, G282, R299, T301, A303, |
| | | S304, G305, V307, K326, D327, G328, W330, |
| | | S343, N344, L345, V346, R347, S348, M349 |
| St. Louis | P09732.2 | S269, E270, P281, G282, R299, T301, A303, |
| encephalitis virus | | S304, G305, L307, K326, D327, G328, W330, |
| | | A343, K344, L345, V346, K347, S348, R349 |
| West Nile virus | Q9Q6P4 | D269, E270, P281, G282, R299, T301, E303, |
| | | S304, G305, L307, D326, S327, G328, W330, |
| | | K343, T344, L345, V346, Q347, S348, G349 |
| Japanese | P27395 | D269, E270, P281, G282, R299, T301, D303, |
| encephalitis virus | | S304, G305, L307, E326, N327, G328, W330, |
| | | T343, T344, L345, V346, R347, S348, Q349 |
| Tick-borne | NP_043135 | K270, Y271, P282, G283, R300, T302, E304, |
| encephalitis virus | | S305, G306, V307, G327, T328, D329, W331, |
| | | G343, G344, L345, V346, R347, S348, M349 |
| Yellow fever | NP_041726.1 | M269, Q270, P281, G282, R299, T301, D303, |
| virus | | S304, G305, V307, S326, D327, G328, W330, |
| | | S343, H344, L345, V346, R347, S348, W349 |
| Usutu virus | AWC68492 | D269, E270, P281, G282, R299, T301, S303, |
| | | S304, G305, L307, K326, N327, G328, W330, |
| | | T343, T344, L345, V346, K347, S348, S349 |
| Powassan virus | ACD88752 | D271, Q272, P283, G284, R301, T303, E305, |
| | | S306, G307, 1309, G328, T329, D330, W332, |
| | | G344, G345, L346, V347, R348, S349, M350 |
| Wesselsbron virus | ABI54474 | H270, N271, P282, G283, R300, T302, D304, |
| | | S305, G306, 1308, P327, D328, G329, W331, |
| | | E343, A344, H345, L346, V347, K348, S349 |

However, the above NS1 amino acid residues from the identified species are merely exemplary, and the invention is not limited to the specific amino acid residues set forth in Table 1. Indeed, in other embodiments, the binding agent may specifically bind to any one or combination of the following NS1 amino acid residues (reference sequence DENV2 (NCBI Accession No. P29990)): K94, G95, I96, T265, G266, P267, W268, G271, K272, L273, F279, C280, T283, G295, P296, S297, L298, T300, T302, K306, I308, T309, W311, R322, Y323, R324, G325, C329, Y331, E340, K341, E342, V350, T351, and/or A352.

In some embodiments, the antigen-binding agent is an antibody, such as a monoclonal antibody, or an antigen-binding fragment thereof. For example, the antigen-binding agent may be a whole antibody. As defined herein, a whole antibody comprises two identical copies of a heavy (H) chain polypeptide and two identical copies of a light (L) chain polypeptide. Each of the heavy chains contains one N-terminal variable (V_{H}) region and three C-terminal constant (C_{H1}, C_{H2}, and C_{H3}) regions, and each light chain contains one N-terminal variable (V_{L}) region and one C-terminal constant (C_{L}) The heavy chain C-terminal constant region contains the fragment crystallizable (Fc) domain, which determines antibody class and is responsible for humoral and cellular effector functions. Antibodies are divided into five major classes (or “isotypes”), IgG, IgM, IgA, IgD and IgE, which differ in their function in the immune system. IgGs are the most abundant immunoglobulins in the blood, representing 60/% of total serum antibodies in humans. IgG antibodies may be subclassified as IgG1, IgG2, IgG3, and IgG4, named in order of their abundance in serum (IgG1 being the most abundant) (Vidarsson et al., Frontiers in Immunology, 5: 520 (2014)). A whole antibody provided herein may be of any suitable class and/or subclass.

As discussed above, an exemplary whole antibody that specifically binds to a flavivirus NS1 protein is denoted “2B7.” 2B7 is an IgG2b mouse monoclonal antibody (mAb) directed against dengue virus NS1 protein and is a strong inhibitor of NS1-induced endothelial hyperpermeability (3). The heavy chain variable region (VII) of the 2B7 antibody comprises an HCDR1 amino acid sequence of SEQ ID NO: 1, an HCDR2 amino acid sequence of SEQ ID NO: 2, and an HCDR3 amino acid sequence of SEQ ID NO: 3. The light chain variable region (VL) of the 2B7 antibody comprises an LCDR1 amino acid sequence of SEQ ID NO: 4, an LCDR2 amino acid sequence of SEQ ID NO: 5, and an LCDR3 amino acid sequence of SEQ ID NO: 6. The heavy chain variable region of 2B7 comprises the amino acid sequence of SEQ ID NO: 7 and the light chain variable region comprises the amino acid sequence of SEQ ID NO: 8.

Thus, in some embodiments, the binding agent disclosed herein comprises three complementarity determining regions (CDRs) of an antibody heavy chain variable region (VH) and three CDRs of an antibody light chain variable region (VL), wherein: (a) CDR1 of the VH (HCDR1) comprises the amino acid sequence of SEQ ID NO: 1, CDR2 of the VH (HCDR2) comprises the amino acid sequence of SEQ ID NO: 2, and CDR3 of the VH (HCDR3) comprises the amino acid sequence of SEQ ID NO: 3; and (b) CDR1 of the LH (LCDR1) comprises the amino acid sequence of SEQ ID NO: 4, CDR2 of the LH (LCDR2) comprises the amino acid sequence of SEQ ID NO: 5, and CDR3 of the LH (LCDR3) comprises the amino acid sequence of SEQ ID NO: 6.

In some embodiments, one or more amino acids of the aforementioned heavy chain variable region, light chain variable region, and CDRs thereof, may be replaced or substituted with a different amino acid. An amino acid “replacement” or “substitution” refers to the replacement of one amino acid at a given position or residue by another amino acid at the same position or residue within a polypeptide sequence. Any suitable number of amino acids may be substituted. In this regard, the aforementioned amino acid sequences may comprise a substitution of one or more amino acids (e.g., 2 or more, 5 or more, or 10 or more amino acids). For example, 1-10 amino acids (e.g., 1, 2, 3, 4, 5, 6, 7, 8, 9, or 10 amino acids) of the aforementioned VH, VL, and/or CDR amino acid sequences may be substituted. In some embodiments, the amino acid substitution is conservative. The terms “conservative amino acid substitution” or “conservative mutation” refer to the replacement of one amino acid by another amino acid with a common physiochemical property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH can be maintained.

In other embodiments, the binding agent comprises an antibody heavy chain variable region and an antibody light chain variable region, wherein the heavy chain variable region comprises the amino acid sequence of SEQ ID NO: 7 and the light chain variable region comprises the amino acid sequence of SEQ ID NO. 8. In other embodiments, the binding agent comprises a CDR amino acid sequence, a heavy chain variable region amino acid sequence, and/or a light chain variable region amino acid sequence that is at least 90% identical (e.g., at least 91%, at least 92%, at least 93%, at least 94%, at least 95%, at least 96%, at least 97%, at least 98%, at least 99%, or 100% identical) to any of the aforementioned amino acid sequences. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

When the binding agent is an antibody, the antibody can be, or can be obtained from, a human antibody, a non-human antibody, or a chimeric antibody as defined herein. A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Fxp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, N.J. (2009)), including, e.g., grafting of non-human CDRs onto a human antibody scaffold (see, e.g., Kashmiri et al., Methods, 36(1): 25-34 (2005); Hou et al., J. Biochem., 144(1): 115-120 (2008); and Strohl, W. R., Strohl, L. M., Therapeutic Antibody Engineering: Current and Future Advances Driving the Strongest Growth Area in the Pharmaceutical Industry (Woodhead Publishing Series in Biomedicine), 1^{st} ed. (2012)).

The antigen-binding agent can also be a fragment or fusion of portions of an antibody, such as any of those defined herein or known in the art (see, e.g., Holliger et al., Nat. Biotech., 23(9): 1126-1129 (2005); and U.S. Pat. No. 9,260,533). In some embodiments, the antigen-binding agent can be a single chain antibody fragment. Examples of single chain antibody fragments include, but are not limited to, (i) a single chain variable fragment (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); and Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen-binding sites. Single chain variable regions have been employed in various therapeutic applications (see, e.g., Strohl, W. R., Strohl, L. M. (eds.), Antibody Fragments as Therapeutics, In Woodhead Publishing Series in Biomedicine, Therapeutic Antibody Engineering, Woodhead Publishing, pp. 265-595 (2012)), and several therapeutic antibody fragments have been approved by the U.S. Food and Drug Administration (FDA).

In some embodiments, the antigen-binding agent is a single-chain variable fragment. A single-chain variable fragment (scFv) is a fusion protein of the variable regions of the heavy (VH) and light chains (VL) of immunoglobulins, connected with a short linker peptide, typically of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. The scFv may be based on or derived from the 2B7 antibody. In this regard, an exemplary scFv comprises a single antibody VH and a single antibody VL, wherein (a) CDR1 of the VH (HCDR1) comprises the amino acid sequence of SEQ ID NO: 1, CDR2 of the VH (HCDR2) comprises the amino acid sequence of SEQ ID NO: 2, and CDR3 of the VH (HCDR3) comprises the amino acid sequence of SEQ ID NO: 3; and (b) CDR1 of the LH (LCDR1) comprises the amino acid sequence of SEQ ID NO: 4, CDR2 of the LH (LCDR2) comprises the amino acid sequence of SEQ ID NO: 5, and CDR3 of the LH (LCDR3) comprises the amino acid sequence of SEQ ID NO: 6. The VH and VL may be joined by any suitable peptide linker known in the art (see, e.g., Huston et al., Proc. Natl Acad. Sci. LISA, 85: 5879-5883 (1988)). For example, the scFv may be engineered to include a linker comprising four repeats of the amino acid sequence GGSG.

The antigen-binding agent may also be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated VH and VL domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The binding agent provided herein is not limited to antibodies or antibody fragments, however. Indeed, the binding agent may be an “alternative protein scaffold” or a fragment thereof. The term “alternative protein scaffold” (also referred to as “antibody mimetic”) refers to a non-antibody polypeptide or polypeptide domain which displays an affinity and specificity towards an antigen of interest similar to that of an antibody. Exemplary alternative scaffolds include a β-sandwich domain such as from fibronectin (e.g., Adnectins), lipocalins (e.g., Anticalin®), a Kunitz domain, thioredoxin (e.g., peptide aptamer), protein A (e.g., AFFIBODY® molecules), an ankyrin repeat (e.g., DARPins), γ-β-crystallin or ubiquitin (e.g., AFFLIN™ molecules), CTLD3 (e.g., Tetranectin), multivalent complexes (e.g., ATRIMER™ molecules or SIMP™ molecules), and AVIMER™ molecules. Alternative protein scaffolds are further described in, for example, Binz et al., Nat. Biotechnol., 23: 1257-1268 (2005); Skerra, Curr. Opin. Biotech., 18: 295-304 (2007); Silverman et al., Nat. Biotechnol., 23: 1493-94 (2005), Silverman et al., Nat. Biotechnol., 24; 220 (2006); Simeon, R. and Chen, Z., Protein Cell., 9(1): 3-14 (2018); and U.S. Patent Application Publication 2009/0181855 A1.

The present disclosure also provides a conjugate comprising the binding agent described herein linked to a therapeutic agent. Antibody-drug conjugates (ADCs, also referred to as “immunoconjugates”) generally are used in the art to target and kill cancer cells; however, more recently ADCs have been generated using antibodies that recognize viral proteins (e.g., structural proteins) that target virus-infected cells (see, e.g., Lacek et al., J Biol Chem., 289(50): 35015-35028 (2014) and Gavrilyuk et al., Journal of Virology, 87(9): 4985-4993 (2013)). Thus, in some embodiments, the conjugate may comprise (1) an antibody, an alternative protein scaffold, or antigen-binding fragments thereof, and (2) a therapeutic protein or non-protein moiety (e.g., an antiviral agent or a cytotoxic agent). Any suitable method know in the art for generating ADCs may be used to generate the aforementioned conjugate (see, e.g., Argwarl, P., Bertozzi, C. R., Bioconjugate Chem., 26(2): 176-192 (2015); Hoffmann et al., Oncoimmunology, 7:3 (2018), DOI: 10.1080/2162402X.2017.1395127; and Yao et al., Int J Mol Sci., 17(2): 194 (2016)).

The disclosure further provides a recombinant NS1 antigen which comprises at least a portion of the NS1 β-ladder domain and at least a portion of the NS1 wing domain. In some embodiments, the recombinant NS1 antigen may comprise two overlapping peptides from the C-terminus β-ladder domain, corresponding to, for example, amino acid residues 260-316 and 288-344 of the DENV2 NS1 full-length amino acid sequence. With respect to the wing domain, the recombinant antigen may comprise the conserved motif W¹¹⁵XXW¹¹⁸G¹¹⁹, which is believed to interact with the cell surface.

The disclosure provides a nucleic acid sequence which encodes the binding agent or the recombinant NS1 antigen described herein, as well as a vector comprising the nucleic acid sequence. The vector can be, for example, a plasmid, a viral vector, phage, or bacterial vector. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994)).

In addition to the nucleic acid encoding the binding agent or recombinant NS1 antigen, the vector desirably comprises expression control sequences, such as promoters, enhancers, polyadenylation signals, transcription terminators, internal ribosome entry sites (IRES), and the like, that provide for the expression of the nucleic sequence in a host cell. Exemplary expression control sequences are known in the art and described in, for example, Goeddel, Gene Expression Technology: Methods in Enzymology, Vol. 185, Academic Press, San Diego, Calif. (1990).

### Compositions and Methods

The disclosure also provides a composition comprising the above-described binding agent, conjugate, recombinant NS1 antigen, or nucleic acid sequences encoding any of the foregoing. The composition desirably is a pharmaceutically acceptable (e.g., physiologically acceptable) composition, which comprises a carrier, preferably a pharmaceutically acceptable (e.g., physiologically acceptable) carrier, and the binding agent, the conjugate recombinant antigen, or nucleic acid sequence. Any suitable carrier can be used within the context of the disclosure, and such carriers are well known in the art. For example, the composition may contain preservatives, such as, for example, methylparaben, propylparaben, sodium benzoate, and benzalkonium chloride. A mixture of two or more preservatives optionally may be used. In addition, buffering agents may be included in the composition. Suitable buffering agents include, for example, citric acid, sodium citrate, phosphoric acid, potassium phosphate, and various other acids and salts. A mixture of two or more buffering agents optionally may be used. Methods for preparing compositions for pharmaceutical use are known to those skilled in the art and are described in, for example, Remington: The Science and Practice of Pharmacy, Lippincott Williams & Wilkins; 21st ed. (May 1, 2005).

One of ordinary skill in the art will appreciate that the composition may comprise other therapeutic or biologically active agents. For example, factors that control inflammation, such as ibuprofen or steroids, can be part of the composition to reduce swelling and inflammation associated with in vivo administration of the composition. To enhance the immune response generated against a flavivirus, the composition also may comprise an immune stimulator, or a nucleic acid sequence that encodes an immune stimulator. Immune stimulators also are referred to in the art as “adjuvants,” and include, for example, cytokines, chemokines, or chaperones. Cytokines include, for example, Macrophage Colony Stimulating Factor (e.g., GM-CSF), Interferon Alpha (IFN-α), Interferon Beta (IFN-β), Interferon Gamma (IFN-γ), interleukins (IL-1, IL-2, IL-4, IL-5, IL-6, IL-8, IL-10, IL-12, IL-13, IL-15, IL-16, and IL-18), the TNF family of proteins, Intercellular Adhesion Molecule-1 (ICAM-1), Lymphocyte Function-Associated antigen-3 (LFA-3), B7-1, B7-2, FMS-related tyrosine kinase 3 ligand, (Flt3L), vasoactive intestinal peptide (VIP), and CD40 ligand. Chemokines include, for example, B Cell-Attracting chemokine-1 (BCA-1), Fractalkine, Melanoma Growth Stimulatory Activity protein (MGSA), Hemofiltrate CC chemokine 1 (HCC-1), Interleukin 8 (IL-8), Interferon-stimulated T-cell alpha chemoattractant (I-TAC), Lymphotactin, Monocyte Chemotactic Protein 1 (MCP-1), Monocyte Chemotactic Protein 3 (MCP-3), Monocyte Chemotactic Protein 4 (CP-4), Macrophage-Derived Chemokine (MDC), a macrophage inflammatory protein (MIP), Platelet Factor 4 (PF4), RANTES, BRAK, eotaxin, exodus 1-3, and the like. Chaperones include, for example, the heat shock proteins Hsp170, Hsc70, and Hsp40.

The disclosure further provides method of inducing an immune response against a flavivirus in a mammal, which comprises administering to the mammal an effective amount of the above-described binding agent, recombinant NS1 antigen, or compositions comprising same, whereupon an immune response against the flavivirus is induced in the mammal. The disclosure also is directed to the use of the above-described binding agent, recombinant NS1 antigen, or compositions comprising same in a method of inducing an immune response against a flavivirus in a mammal. The immune response can be a humoral immune response, a cell-mediated immune response, or, desirably, a combination of humoral and cell-mediated immunity. Ideally, the immune response provides protection upon subsequent challenge with a flavivirus of any type. However, protective immunity is not required in the context of the invention. The inventive method further can be used for antibody production and harvesting in non-human mammals (e.g., rabbits or mice).

Any route of administration can be used to deliver the binding agent, conjugate, recombinant NS1 antigen, or composition to the mammal. Indeed, although more than one route can be used to administer the composition, a particular route can provide a more immediate and more effective reaction than another route. Preferably, the composition is administered via intramuscular injection or intranasal administration. The composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The dose of binding agent or recombinant NS1 antigen included in the composition administered to the mammal will depend on a number of factors, including the age and gender of the mammal, the extent of any side-effects, the particular route of administration, and the like. The dose ideally comprises an “effective amount” of binding agent or recombinant NS1 antigen, i.e., a dose which provokes a desired immune response in the mammal. The desired immune response can entail production of antibodies, protection upon subsequent challenge, immune tolerance, immune cell activation, and the like. Preferably, the desired immune response results in sufficient immunity for the recipient for a desired period of time such that subsequent infection with any other flavivirus does not result in illness.

Administering the composition containing the binding agent or the recombinant NS1 antigen can be one component of a multistep regimen for inducing an immune response against a flavivirus in a mammal. In particular, the inventive method can represent one arm of a prime and boost immunization regimen. In this respect, the method comprises administering to the mammal a boosting composition after administering the composition comprising the binding agent, the conjugate, or the recombinant NS1 antigen to the mammal. In such embodiments, therefore, the immune response is “primed” upon administration of the composition containing the binding agent or the recombinant NS1 antigen and is “boosted” upon administration of the boosting composition. The boosting composition may also comprise the binding agent or the recombinant NS1 antigen.

Administration of the priming composition and the boosting composition can be separated by any suitable timeframe, e.g., 1 week or more, 2 weeks or more, 4 weeks or more, 8 weeks or more, 12 weeks or more, 16 weeks or more, 24 weeks or more, 52 weeks or more, or a range defined by any two of the foregoing values. The boosting composition desirably is administered to a mammal (e.g., a human) 2 weeks or more (e.g., 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 20 weeks, 24 weeks, 28 weeks, 35 weeks, 40 weeks, 50 weeks, 52 weeks, or a range defined by any two of the foregoing values) following administration of the priming composition. More than one dose of priming composition and/or boosting composition can be provided in any suitable timeframe. The dose of the priming composition and boosting composition administered to the mammal depends on a number of factors, including the extent of any side-effects, the particular route of administration, etc.

The binding agent, conjugate, recombinant NS1 antigen, compositions comprising any of the foregoing, and components thereof can be provided in a kit, e.g., a packaged combination of reagents in predetermined amounts with instructions for performing a method using the binding agent, conjugate, recombinant NS1 antigen, or composition. As such, the disclosure provides a kit comprising the binding agent, conjugate, recombinant NS1 antigen, or composition described herein and instructions for use thereof. The instructions can be in paper form or computer-readable form, such as a disk, CD, DVD, etc. Alternatively or additionally, the kit can comprise a calibrator or control, and/or at least one container (e.g., tube, microtiter plates, or strips) for conducting a method, and/or a buffer. Ideally, the kit comprises all components, i.e., reagents, standards, buffers, diluents, etc., which are necessary to perform the method. Other additives may be included in the kit, such as stabilizers, buffers (e.g., a blocking buffer or lysis buffer), and the like. The relative amounts of the various reagents can be varied to provide for concentrations in solution of the reagents which substantially optimize the method. The reagents may be provided as dry powders (typically lyophilized), including excipients which on dissolution will provide a reagent solution having the appropriate concentration.

The following examples further illustrate the invention but, of course, should not be construed as in any way limiting its scope.

### Example 1

This example demonstrates that the anti-DENV NS1 IgG2b mouse monoclonal antibody (mAb) 2B7 protects a mouse model against lethal dengue virus infection and NS1-mediated vascular leak and endothelial dysfunction.

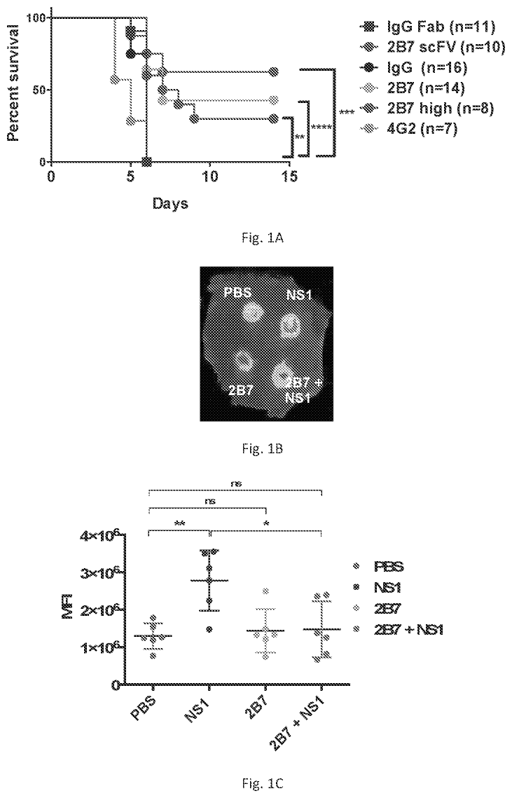

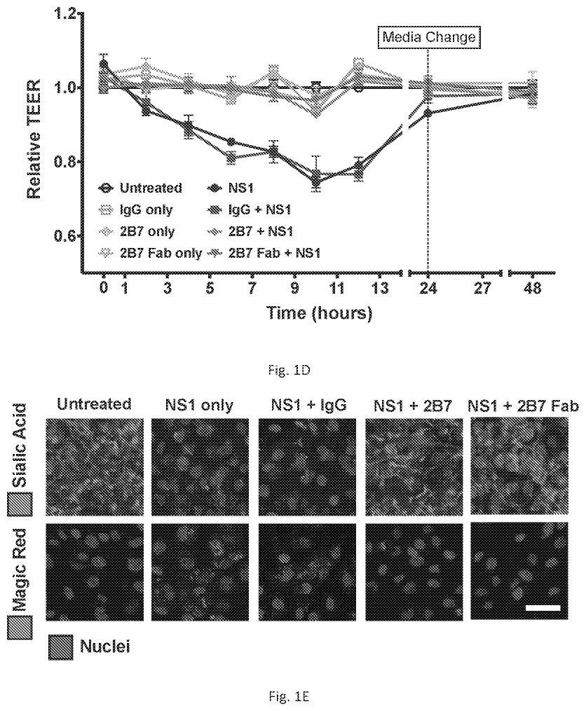

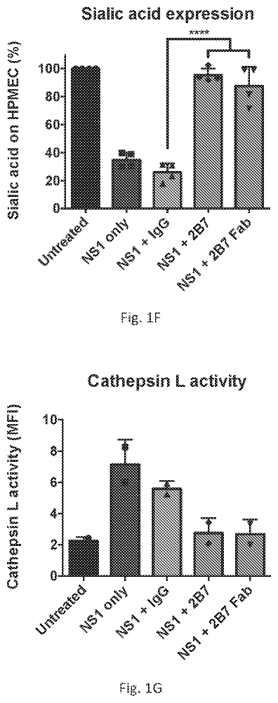

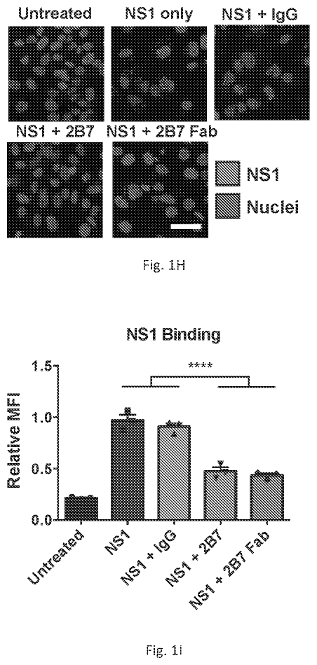

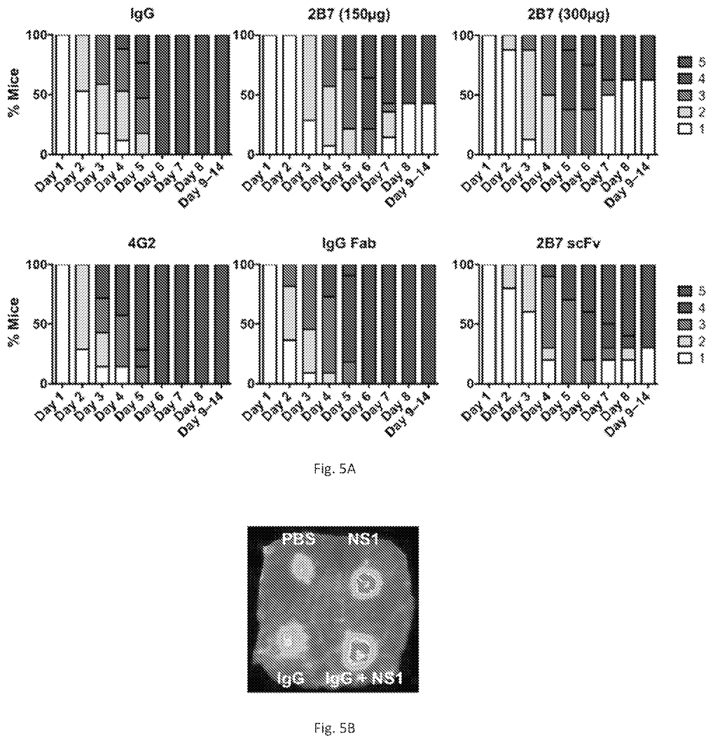

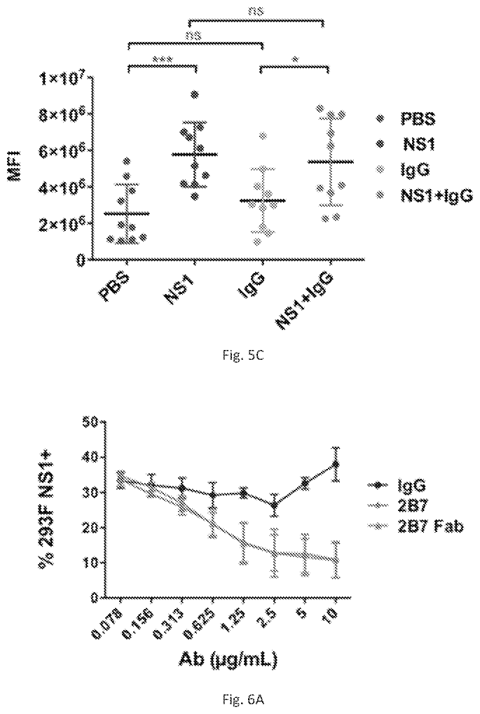

The anti-DENV NS1 IgG2b mouse monoclonal antibody (mAb) 2B7 was previously identified as a strong inhibitor of NS1-induced endothelial hyperpermeability (3). In a DENV2 lethal challenge mouse model, 2B7 was protective in a dose-dependent manner compared to an IgG isotype control, as was a single chain variable fragment (scFv) of 2B7, suggesting that protection could be achieved in a manner independent of antibody Fc effector functions (FIG. 1A and FIG. 5A). In contrast, an anti-E antibody (4G2) given at the same dose was not protective, and in fact led to an accelerated time to death (FIG. 1A). Further, 2B7 blocked DENV NS1-mediated vascular leak in the mouse dermis compared to an IgG isotype control (FIG. 1B, FIG. 1C, FIG. 5B, and FIG. 5C).

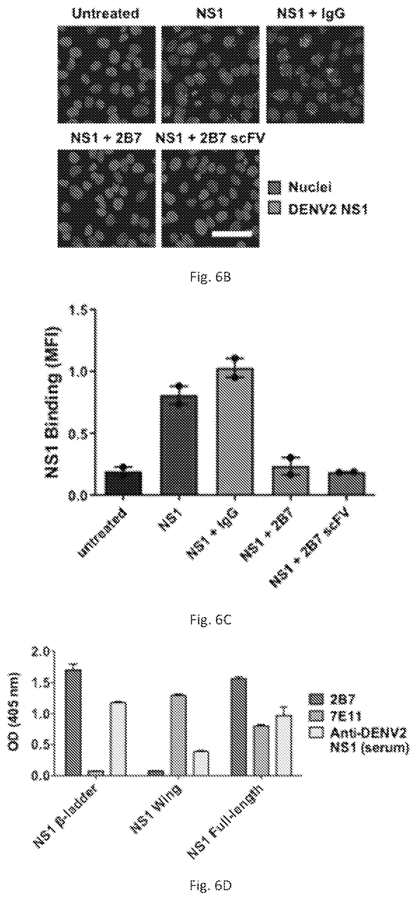

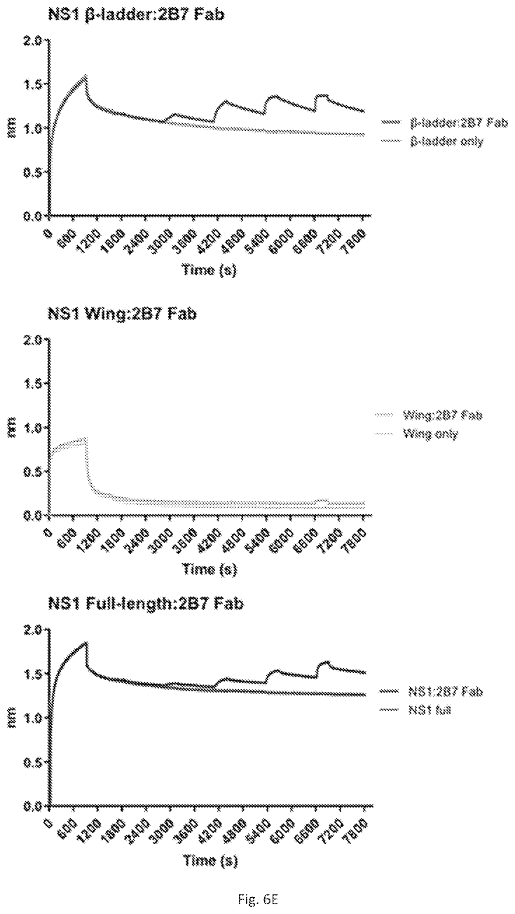

Next, the protective mechanism of 2B7 was investigated in vitro using human pulmonary microvascular endothelial cells (HPMEC) and measuring electrical resistance in a trans-endothelial electrical resistance (TEER) assay. Both 2B7 and its antigen-binding fragment (Fab), but not an IgG isotype control, were sufficient to abrogate NS1-induced endothelial hyperpermeability (FIG. 1D). In addition, 2B7 and its Fab were sufficient to abrogate NS1-mediated endothelial dysfunction of HPMEC measured via cathepsin L activation and disruption of the EGL (measured via surface levels of sialic acid) (13, 26) (FIGS. 1F-1G). It was also determined that 2B7, as well as its Fab and scFv, were sufficient to block binding of NS1 to HPMEC and 293F cells (FIGS. 1H and 1I, and FIGS. 6A-6C).

### Example 2

This example demonstrates that the crystal structure of the 2B7 antigen-binding fragment complexed with DENV NS1 reveals binding to the β-ladder domain.

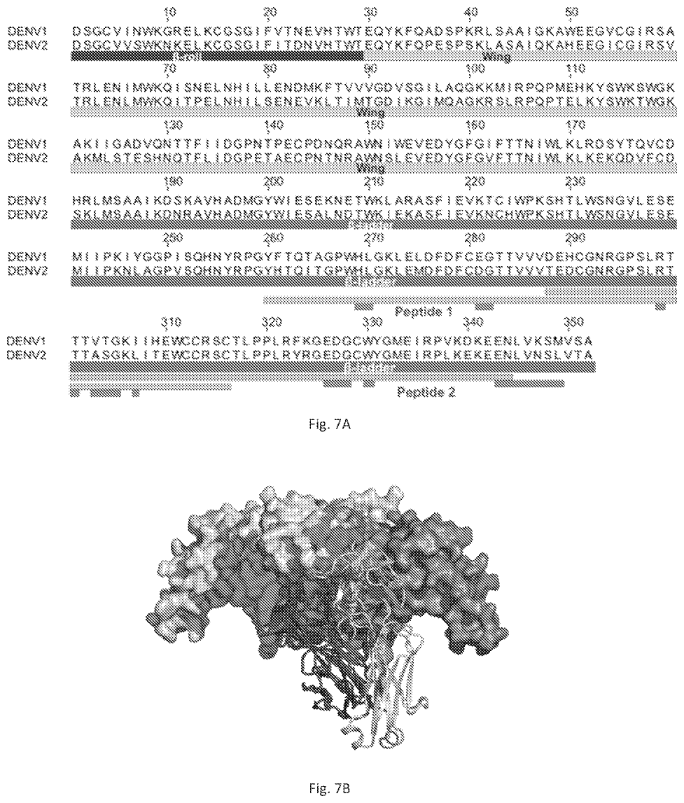

As discussed above, NS1 has three distinct domains: N-terminal pi-roll, wing, and C-terminal β-ladder (21). An ELISA measuring binding of 2B7 to full-length NS1, a recombinant wing domain (residues 38-151 of SEQ ID NO: 9), or a recombinant β-ladder domain (residues 176-352 of SEQ ID NO: 9) indicated that 2B7 bound strongly to both full-length NS1 and the β-ladder, but not to the wing domain (FIG. 6D). This observation was confirmed using biolayer interferometry (BLI) (FIG. 6E), which also revealed NS1 binding K_{d} values of 4.8±3.1 nM for 2B7 full-length, 8.3±6.8 nM for the 2B7 Fab, and 5.8±1.1 nM for the 2B7 scFv. The VirScan phage display system with 56-mer overlapping peptides tiled across the DENV2 NS1 polypeptide was used to identify the epitope target region. 2B7 was found to interact with two overlapping peptides from the C-terminal β-ladder domain (residues 260-316 and 288-344 of SEQ ID NO: 9), consistent with the ELISA and BLI results (27) (FIGS. 7A and 7B).

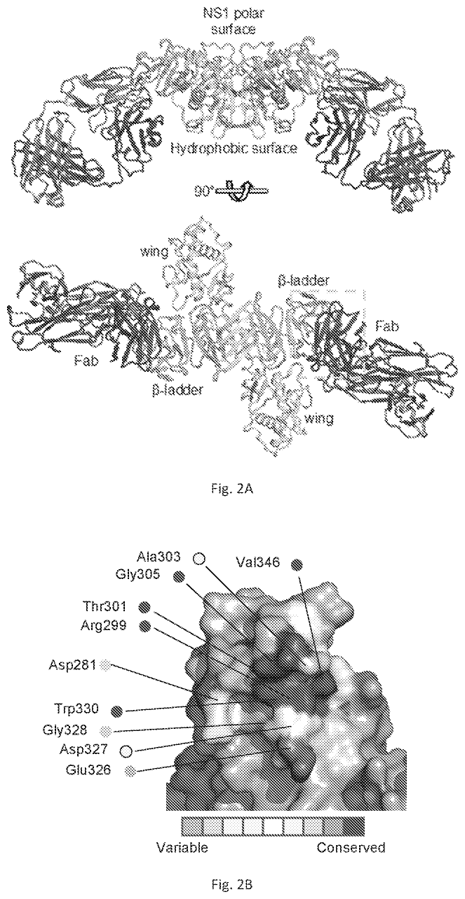

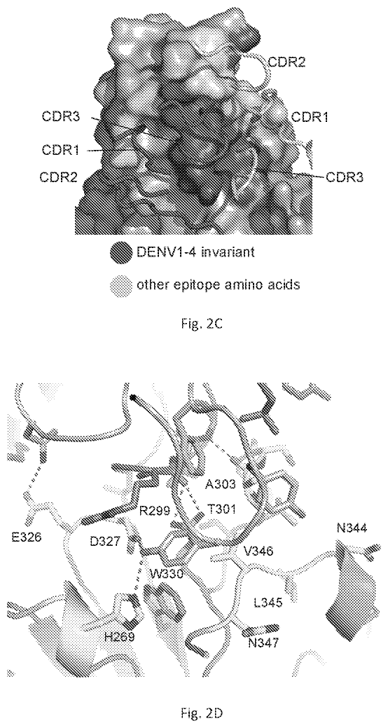

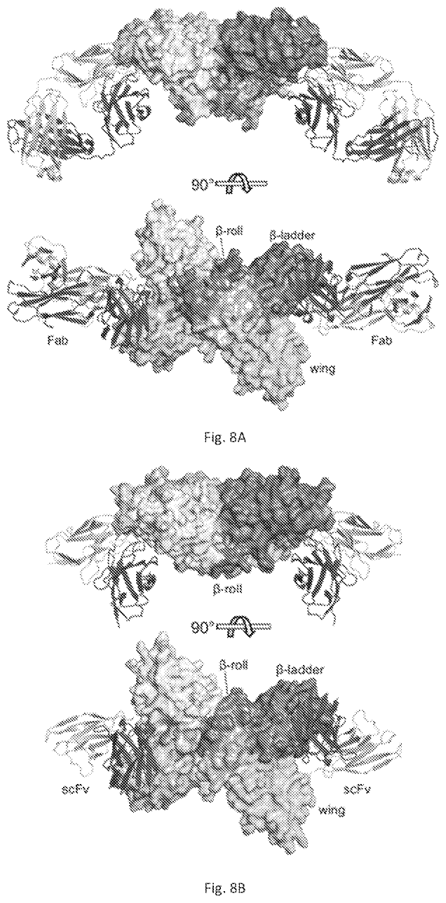

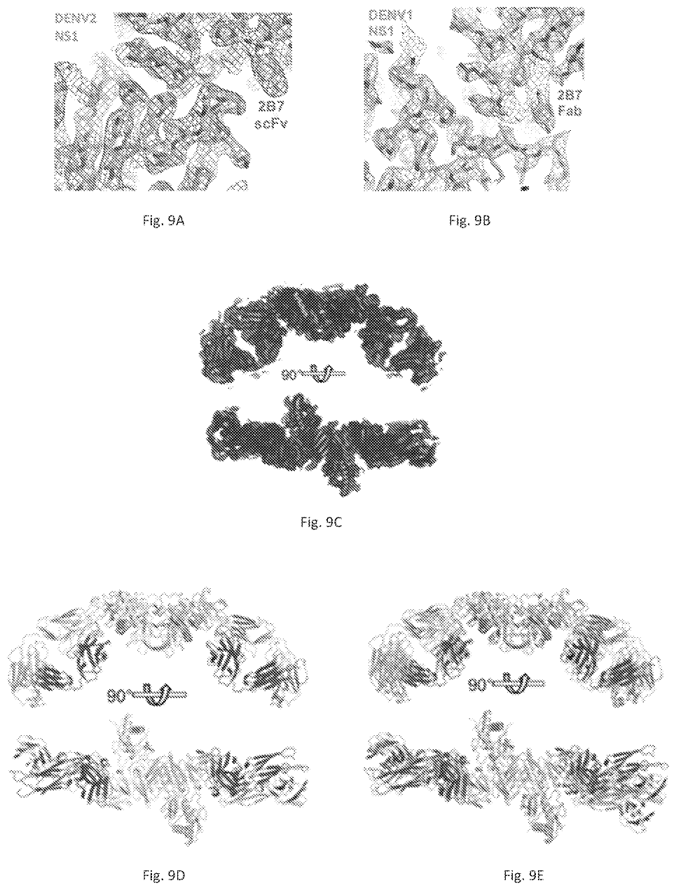

The interaction between 2B7 and NS1 was visualized in detail by solving crystal structures of the 2B7 Fab and scFv in complex with DENV1 NS1 (Fab 3.3 Å, FIG. 2A, FIG. 7B) and DENV2 NS1 (scFv-2.89 Å, FIG. 2B-D, FIG. 8B, FIG. 9A; Fab 4.2 Å, FIGS. 9C and 9D; and Table 2).

TABLE 2

| Diffraction data | DENV2 NS1:2B7 scFv | | DENV1 NS1:2B7 Fab | | DENV2 NS1:2B7 Fab | |
|---|---|---|---|---|---|---|
| Wavelength (Å) | 1.0332 | | 1.0332 | | 1.0332 | |
| Data range (Å) | 47.38-2.89 | | 48.17-3.2 | | 29.80-3.96 | |
| (inner, outer shells) | (47.38-6.2, 2.99-2.89) | | (48.17-8.15, 3.31-3.20) | | (29.80-8.58, 4.10-3.96) | |
| Space group | P2₁2₁2₁ | | P2₁ | | I4₁22 | |
| Unit cell | 69.63 165.59 258.59 | | 67.45 329.71 86.72 | | 148.47 148.47 517.05 | |
| | 90 90 90 | | 90 90.78 90 | | 90 90 90 | |
| Unique reflections | 67,710 | (7177, 6307) | 62,008 | (3790, 6154) | 25,742 | (2732, 2415) |
| Multiplicity | 13.2 | (12.3, 13.0) | 7.1 | (6.9, 6.9) | 26.6 | (23.5, 24.5) |
| Completeness (%) | 99.34 | (99.7, 94.33) | 99.87 | (99.79, 99.97) | 98.81 | (99.96, 95.34) |
| Mean I/sigma(I) | 11.41 | (30.0, 1.18) | 5.9 | (18.9, 0.9) | 13.8 | (52.3, 0.9) |
| R-merge | 0.198 | (0.069, 2,319) | 0.300 | (0.075, 1.661) | 0.215 | (0.049, 4.703) |
| R-pim | 0.057 | (0.653) | 0.122 | (0.031, 0.6778) | 0.042 | (0.010, 0.9601) |
| CC_{1/2} | 0.998 | (0.998, 0.532) | 0.992 | (0.998, 0.415) | 1 | (1, 0.703) |

Refinement

| # reflections | 67,534 | 61,969 | | 25,540 | |
|---|---|---|---|---|---|
| # reflections R-free | 2412 | 3,092 | | 1,276 | |
| R-work | 0.223 | 0.207 | | 0.266 | |
| R-free | 0.269 | 0.262 | | 0.306 | |
| CC-work | 0.925 | 0.932 | (0.610) | 0.928 | (0.614) |
| CC-free | 0.916 | 0.822 | (0.550) | 0.795 | (0.500) |

| | | | |
|---|---|---|---|
| # non-hydrogen atoms | 17,581 | 24,041 | 11,441 |
| protein | 17,525 | 23,924 | 11,441 |
| sugar | 56 | 117 | — |
| # amino acid residues | 2248 | 3081 | 1491 |
| RMSD (bonds, Å) | 0.010 | 0.011 | 0.003 |
| RMSD (angles, °) | 1.20 | 1.24 | 0.66 |
| Ramachandran favored (%) | 94.78 | 94.18 | 95.05 |
| allowed (%) | 4.90 | 5.79 | 4.74 |
| outliers (%) | 0.32 | 0.03 | 0.21 |
| Rotamer outliers (%) | 6.0 | 7.3 | 6.9 |
| Clashscore | 9.1 | 10.6 | 9.6 |
| Average B-factor (Å²) | 84.7 | 90.1 | 244.1 |
| macromolecules | 84.7 | 90.1 | 244.1 |
| sugar | 90.1 | 103.8 | — |
| # TLS groups | 39 | 12 | 18 |
| PDB code | 7K93 | 6WEQ | 6WER |

Each NS1 dimer binds two copies of the scFv/Fab fragment—one to each distal tip of the β-ladder (FIG. 2A, FIG. 8, FIGS. 9C-9E). The NS1:2B7 complex has an overall arch shape, where the antibody fragments form the sides of the arch, with the membrane-facing hydrophobic side of NS1 on the arch inner surface (FIG. 2A, FIG. 8). In this configuration, the 2B7 Fab would likely prevent the hydrophobic face of NS1 from interacting with cell surfaces. The 2.89-Å electron density map was of sufficient quality to confidently build the scFv constant and variable regions as well as the side chains of the combining site of the 2B7 scFV and the DENV2 NS1 discontinuous epitope (FIG. 2B-D, FIG. 9A). The variable loops of the 2B7 light chain make numerous contacts with NS1 β-ladder residues, consistent with the ELISA, BLI, and phage display results (FIG. 2B-D, FIGS. 7-9, FIG. 9F). The binding modalities of 2B7 to NS1 were identical in the three structures (FIG. 9E), and the NS1 structure was unchanged by 2B7 binding.

### Example 3

This example demonstrates that the 2B7 antibody is cross-reactive with NS1 from multiple flavivirus species.

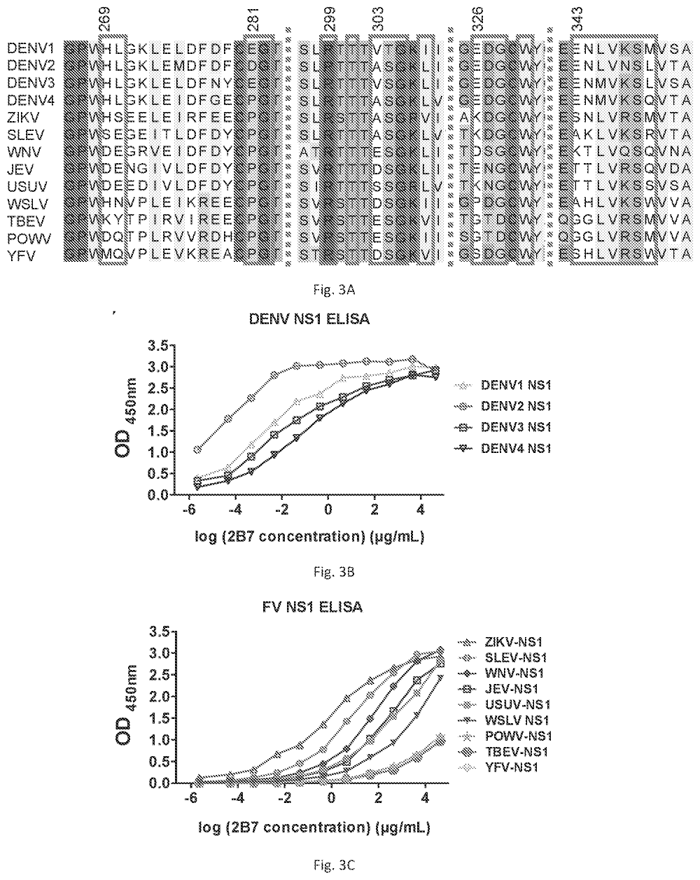

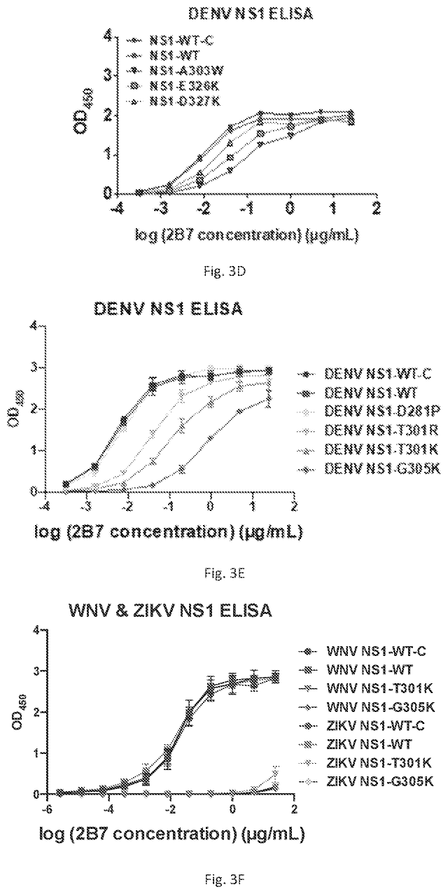

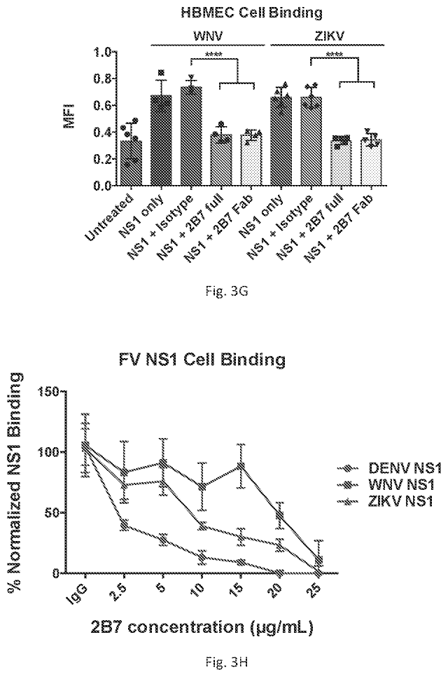

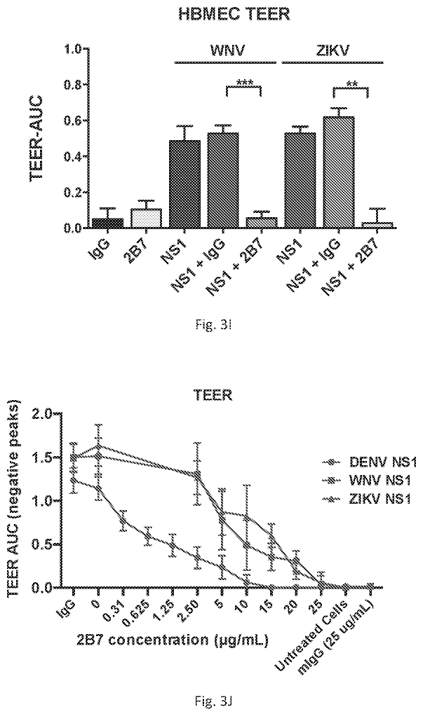

The amino acid residues in the 2B7 epitope can be divided into two classes: the epitope core region, composed of residues that are highly conserved across flaviviruses, and the epitope periphery, displaying varying levels of divergence among flaviviruses. (FIGS. 2B-D and 3A). To test the hypothesis that 2B7 would recognize distinct flavivirus NS1 proteins with affinities that correlated with the extent of conservation with DENV NS1, ELISAs were performed to measure the relative affinities of 2B7 for a panel of flavivirus NS1 proteins including DENV1-4, ZIKV, Saint Louis encephalitis virus (SLEV), WNV, JEV, tick-borne encephalitis virus (TBEV), Powassan virus (POWV), Usutu virus (USUV), Wesselsbron virus (WSLV), and yellow fever virus (YFV). 2B7 bound most tightly to NS1 from DENV1-4, followed by ZIKV, SLEV, WNV, JEV, USUV, and WSLV, with minimal binding detected for YFV, POWV, and TBEV. The strength of binding correlated with the degree of conservation with DENV NS1 (FIGS. 3B and C). To determine the relative contribution of key flavivirus NS1 amino acid residues on 2B7 binding, single, double, triple, or quadruple amino acid substitutions of the DENV2 NS1 β-ladder amino acids were introduced within the NS1:2B7 epitope (Table 3).

TABLE 3

| | | | | |
|---|---|---|---|---|
| DE | WT | ++ | +++ | 0.006 |
| | D281P | ++ | +++ | 0.008 |
| | R299E | − | ND | |
| | R299A | − | ND | |
| | T301K | ++ | + | 0.130 |
| | T301R | + | ++ | 0.041 |
| | A303W | ++ | + | 0.120 |
| | A303R | ++ | + | |
| | G305K | ++ | + | 0.806 |
| | G305W | + | + | |
| | E326K | ++ | + | 0.040 |
| | E326A | ++ | ++ | |
| | D327K | ++ | ++ | 0.018 |
| | G328K | + | + | |
| | G328W | + | + | |
| | W330A | − | ND | |
| | V346E | ++ | ++ | |
| | R299E V346E | − | ND | |
| | E326K E327K | + | + | |
| | R299E E326K E327K | − | ND | |
| | R299E E326K V346E | − | ND | |
| | R299E D327K V346E | − | ND | |
| | E326K D327K V346E | + | + | |
| | R299E E326K E327K V346E | − | ND | |
| WNV | WT | ++ | ++ | 0.018 |
| NS1 | T301K | ++ | − | N/A |
| | G305K | ++ | − | N/A |
| ZIKV | WT | ++ | ++ | 0.014 |
| | T301K | ++ | − | N/A |
| | G305K | ++ | − | N/A |

Mutant NS1 proteins produced alongside a summary of secretion and 2B7-binding properties from the ELISA data. For secretion, a ″++″ represents secretion comparable to wild-type (WT), ″+″ represents any level of detectable protein in the supernatant but significantly less than WT, while ″−″ represents undetectable levels of protein in the supernatant. For 2B7 binding, NS1-WT binding levels are indicated as ″+++″, and levels of NS1-mutant binding are compared relative to this level. ND represents mutants not tested for 2B7 binding because of secretion defects. ″−″ represents mutants with minimal binding to 2B7, 2B7 EC50 values were calculated only for NS1 mutants analyzed by ELISA in FIG. 3 D-F. Mutants for which 2B7 EC50 could not be calculated are indicated by ″N/A″.

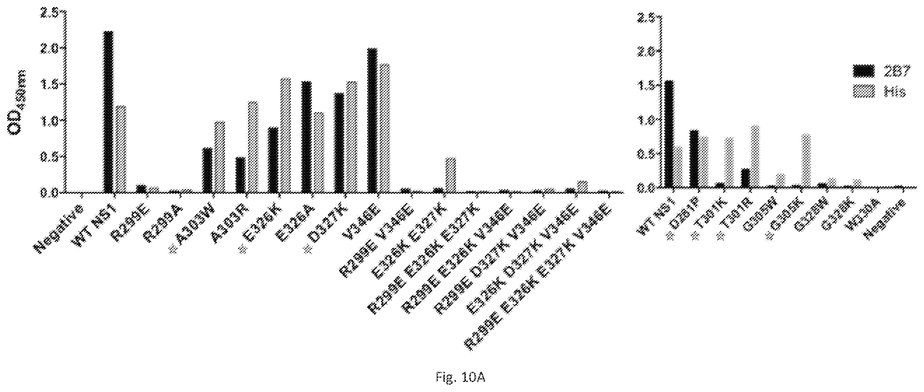

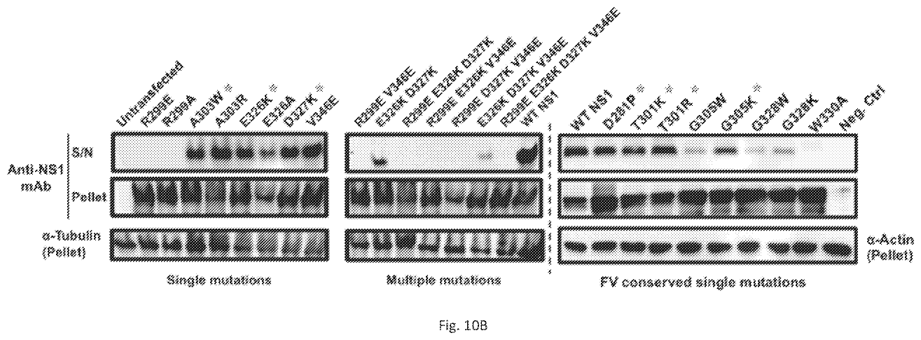

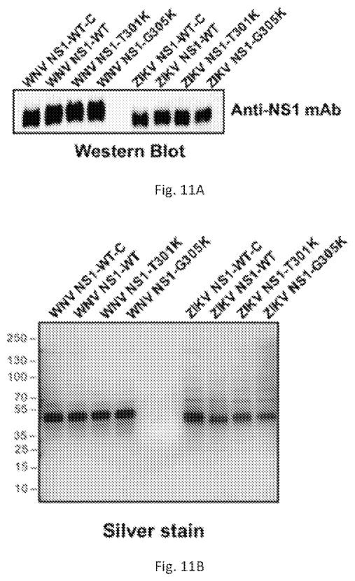

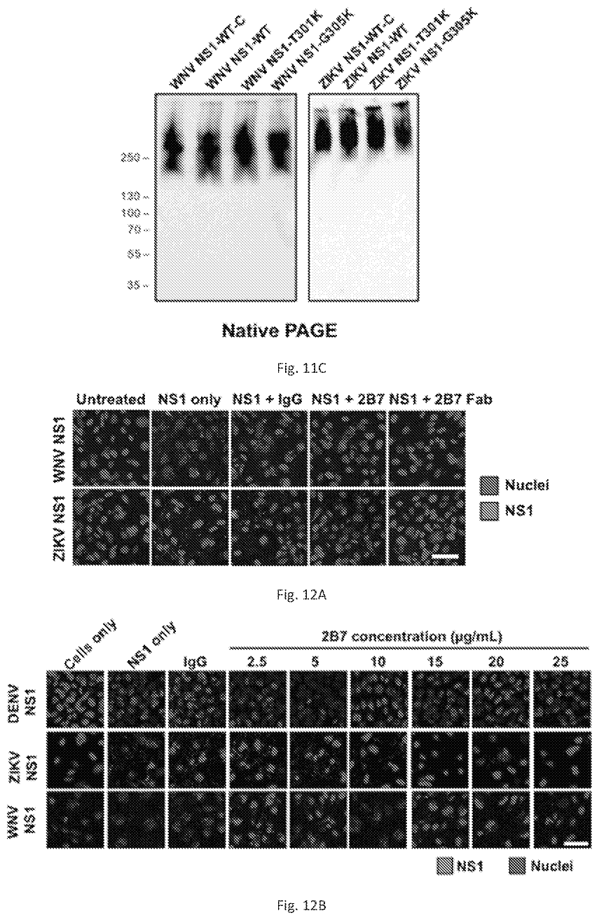

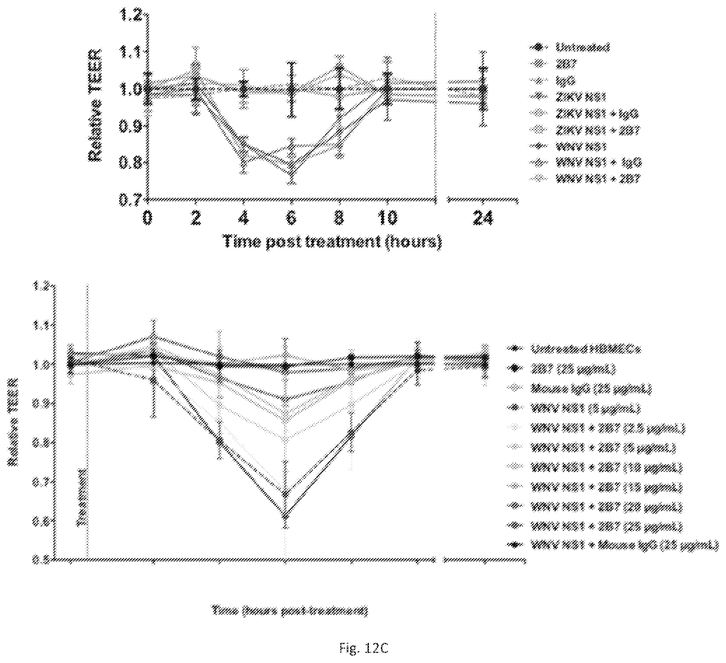

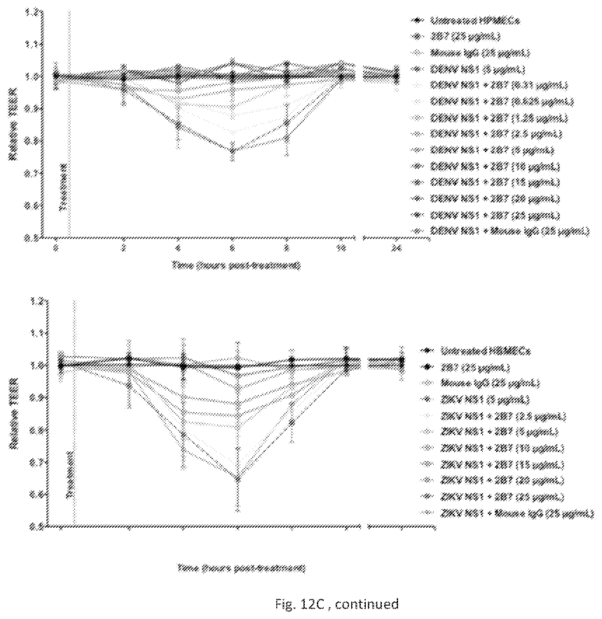

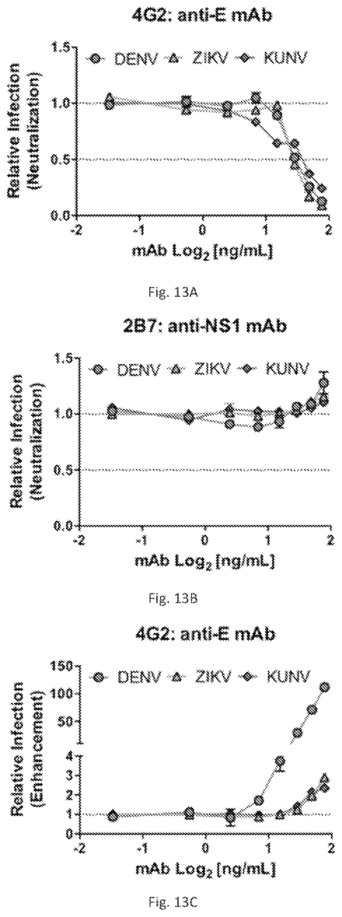

NS1 mutants produced in 293T cells were initially screened for candidates that were secreted and displayed diminished binding to 2B7 (FIGS. 10A and 10B). Seven DENV NS1 β-ladder single substitutions were then selected (NS1-D281P, NS1-T301R, NS1-T301K, NS1-A303W, NS1-G305K, NS1-E326K, and NS1-D327K) for purification and 2B7 binding ELISAs. A direct ELISA revealed that, with the exception of NS1-D281P, each of these mutants displayed weaker binding to 2B7 compared to NS1-WT (FIGS. 3D and E, FIG. 10A, Table 3). To confirm the role of the flavivirus-conserved residues above in 2B7 binding to multiple flavivirus NS1 proteins, two single mutants were created for both WNV and ZIKV NS1 proteins (WNV/ZIKV NS1-T301K and NS1 G305K) (FIGS. 11A-11C). A direct ELISA revealed that these NS1 mutants exhibited a severe binding defect to 2B7 compared to their respective NS1-WT control proteins (FIG. 3F). To test if the NS1 cross-reactivity of 2B7 correlated with function, ZIKV and WNV NS1 proteins were used in NS1-cell binding and TEER assays on human brain microvascular endothelial cells (HBMEC). 2B7, but not an IgG isotype control, blocked binding and abrogated NS1-mediated endothelial hyperpermeability in a dose-dependent manner correlating with conservation to DENV2 NS1, indicating that one cross-reactive NS1 monoclonal antibody could inhibit pathogenic functions of NS1 from multiple flaviviruses (FIG. 3G-J, FIGS. 12A-12C). As ADE of DENV infection is problematic for anti-E antibodies, anti-NS1 mAb 2B7 was confirmed to be non-neutralizing and also incapable of mediating ADE of DENV, ZIKV, and the WNV strain Kunjin virus (KUNV) infection (FIGS. 13A-13D). Further, as anti-NS1 antibodies have been reported to modulate the clotting cascade (28) as well as bind to endothelial cells, which may directly mediate endothelial dysfunction (29), the capacity of 2B7 to alter clotting time of human plasma and to bind to the surface of endothelial cells was tested.

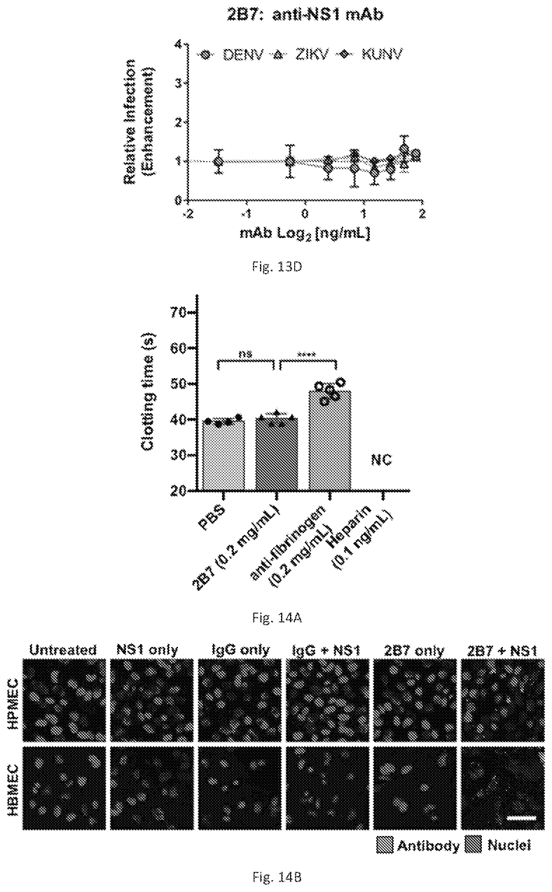

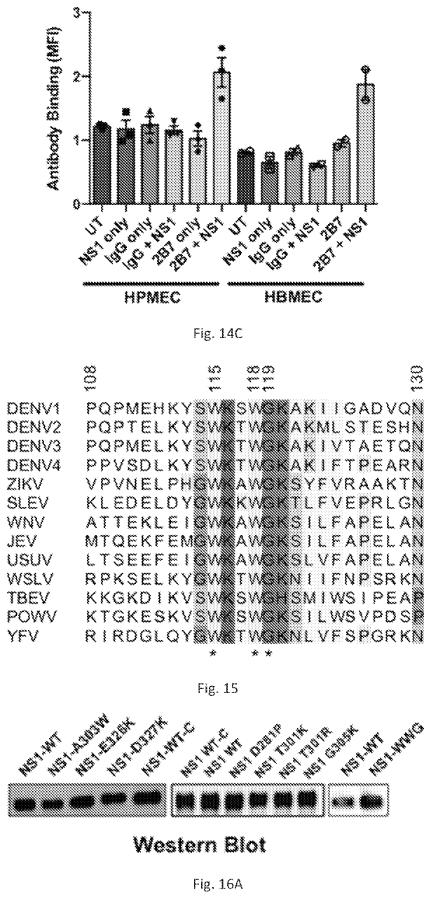

These experiments determined that 2B7 does not alter human clotting time and binds to the surface of endothelial cells significantly more than an isotype control only when NS1 is present, suggesting that 2B7 would likely not enhance DENV disease through these specific mechanisms (FIGS. 14A-14C).

### Example 4

This example describes an investigation of the molecular basis of NS1-mediated endothelial dysfunction.

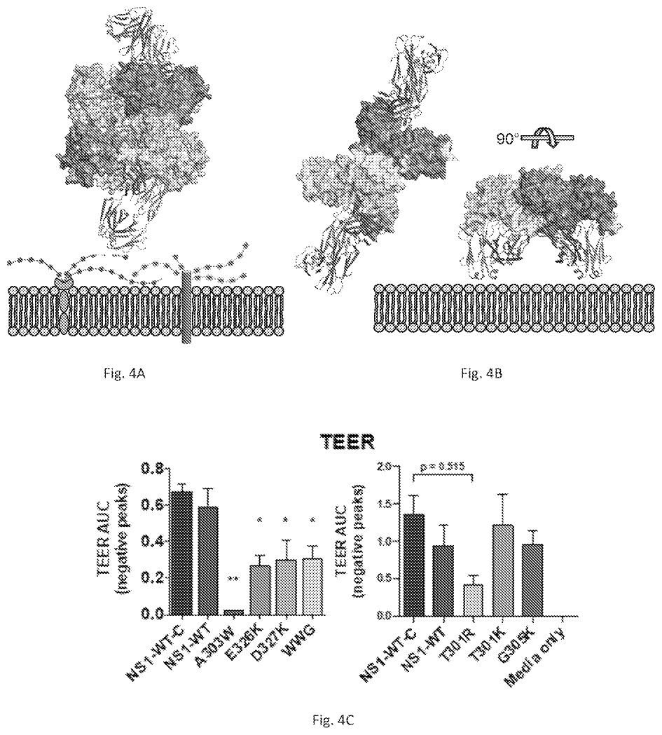

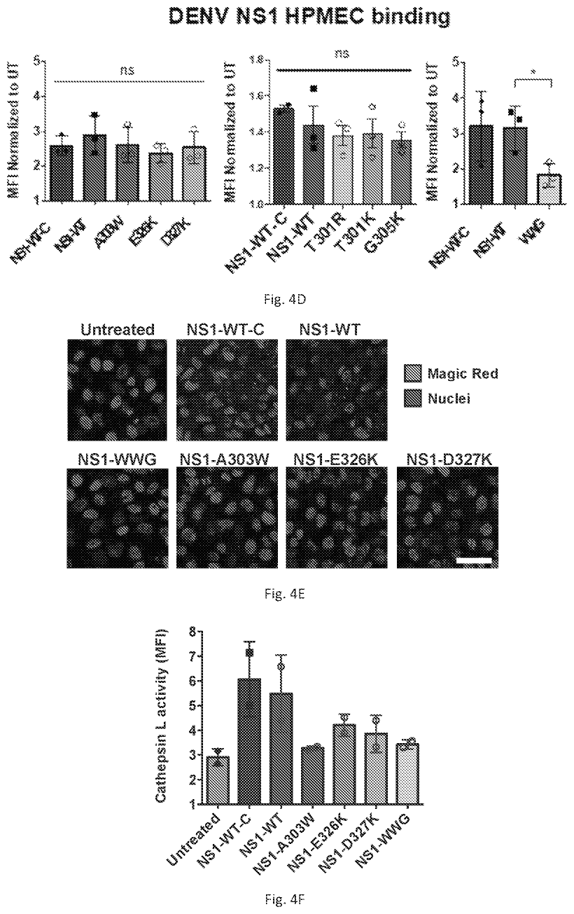

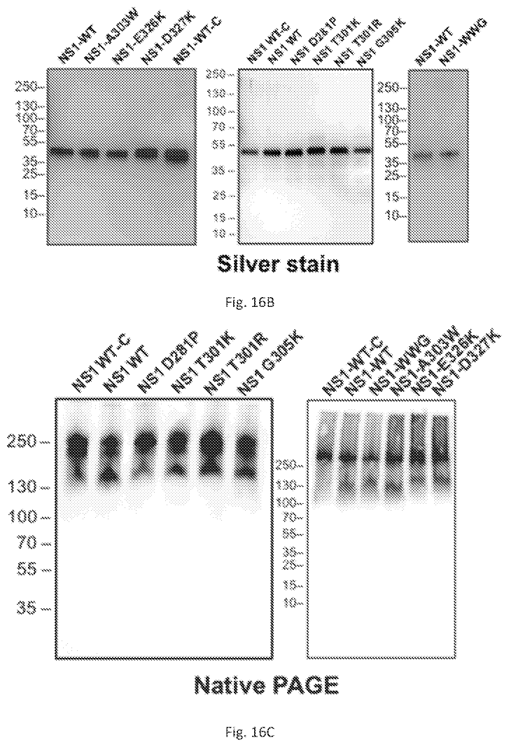

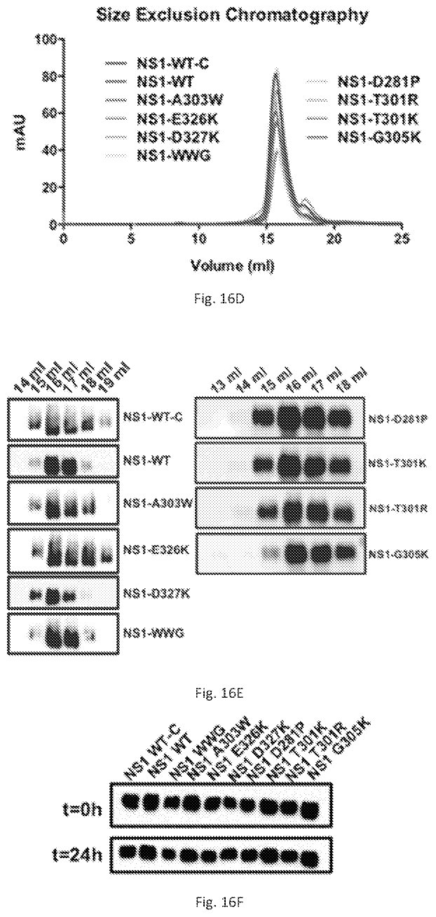

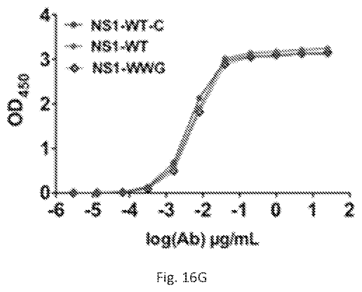

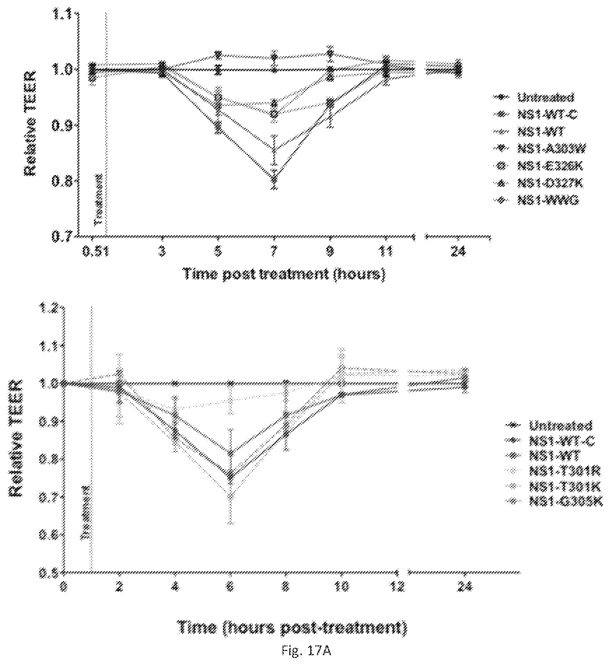

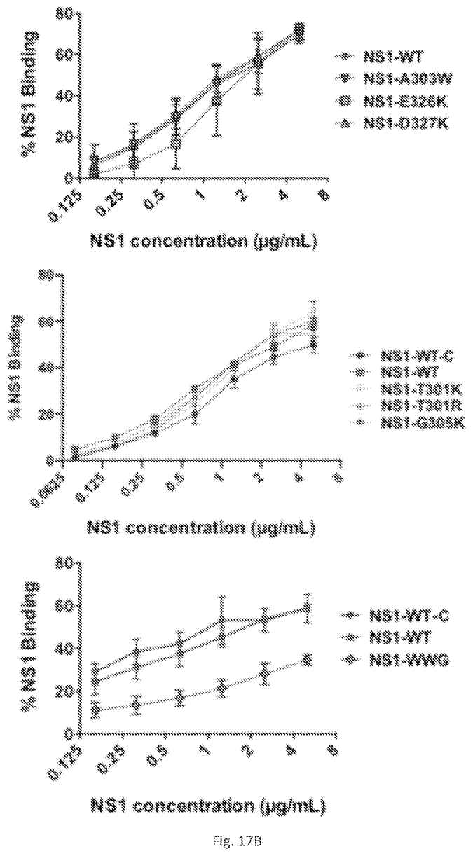

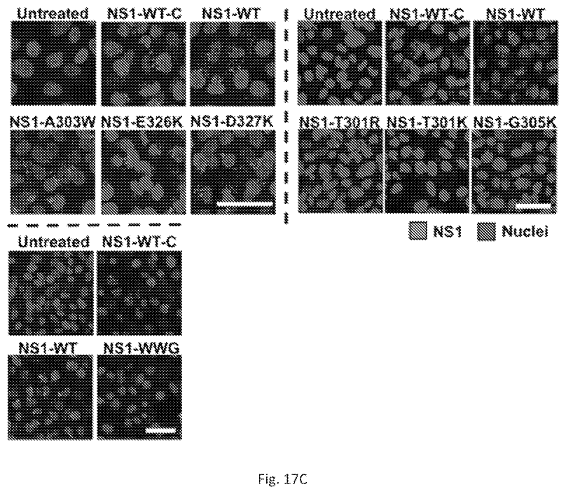

As NS1 is reported to mediate endothelial dysfunction through distinct steps including cell binding, EGL disruption, and endothelial hyperpermeability (13, 14, 16), the mode of 2B7 binding to NS1 provides an opportunity to investigate the molecular basis of NS1-mediated endothelial dysfunction. Although 2B7 binds to the β-ladder, its tilted orientation towards the NS1 hydrophobic surface (FIG. 2A, FIG. 8, FIG. 9, and FIGS. 4A and 4B) predicts that 2B7 would create indirect steric hinderance for the wing domain and would interfere with a predicted interaction between the NS1 wing domain and the cell surface, whether NS1 is in the dimeric or hexameric form (22, 30) (FIGS. 4A-4B). As such, 2B7 was predicted to interfere with both the wing (indirect steric hinderance) and β-ladder (direct binding) of NS1, suggesting that both domains may be critical for NS1 pathology. To test this prediction, mutant expression constructs for NS1 were generated with substitutions predicted to hinder binding to the cell surface or to 2B7. In addition to the DENV NS1 β-ladder mutants described above, a DENV NS1 triple mutant was created having substitutions within the flavivirus-conserved W¹¹⁵XXW¹¹⁸G¹¹⁹ motif (A¹¹⁵XXA¹¹⁸A¹¹⁹) in an immunodominant region of the wing, which is predicted to interact with the cell surface (22, 30-32) (FIGS. 4A-4B and FIG. 15). Six DENV NS1 β-ladder single substitution mutants were purified (NS1-T301R, NS1-T301K, NS1-A303W, NS1-G305K, NS1-E326K, and NS1-D327K) along with the wing domain triple substitution mutant (NS1-WWG>AAA, “NS1-WWG”). All proteins were expressed, secreted, oligomeric, stable, and of purity comparable to wild-type NS1 (NS1-WT) (FIGS. 16A-16F). Further, in contrast to the NS1 β-ladder mutants, the wing domain mutant did not exhibit diminished binding to 2B7 compared to NS1-WT (FIGS. 3D-3E, and FIG. 16G). Intriguingly, in a TEER assay on HPMEC with these mutant NS1 proteins, all NS1 mutants except the β-ladder mutants NS1-D301K and NS1-G305K were defective in their capacity to mediate endothelial hyperpermeability, as compared to NS1-WT, with NS1-A303W displaying the greatest functional defect (FIG. 4C and FIG. 17A). These data indicate that several residues both in the tip of the β-ladder and separately, in the wing domain, are critical for NS1-mediated endothelial dysfunction. To investigate the mechanism(s) of the functional defects of these NS1 mutants, an NS1 cell-binding assay was conducted using both HPMEC and 293F cells, which showed that NS1 with β-ladder substitutions bound to cells comparably to NS1-WT, but NS1-WWG possessed a significant cell binding defect (FIG. 4D and FIGS. 17B-17C). In contrast, when steps downstream of NS1 binding were examined, such as activation of cathepsin L, all mutants were defective relative to NS1-WT (focusing only on the DENV conserved β-ladder mutants) (FIGS. 4E and F).

Taken together, these data show that the NS1 wing domain, specifically the WWG motif, is important for initial attachment of NS1 to cells, whereas the tip of the β-ladder is critical for downstream events required for NS1-mediated endothelial dysfunction.

The above-described structural investigation of the protective mechanism of the 2B7 mAb against DENV infection and DENV NS1-mediated vascular leak in vivo, as well as pan-flavivirus NS1-triggered endothelial dysfunction in vitro, serves as a proof-of-concept that one cross-reactive antibody targeting flavivirus NS1 proteins can provide protection against NS1-mediated pathology from multiple flaviviruses. Further, the structure of the 2B7 scFv/Fab in complex with NS1 revealed that 2B7 obscures the β-ladder through direct binding and the wing domain through indirect steric hinderance of the NS1 dimer and/or hexamer form, suggesting that one anti-NS1 mAb can simultaneously antagonize the cellular interactions of two domains. DENV NS1 mutagenized in these domains revealed the importance of these domains in NS1-mediated endothelial dysfunction, implicating the wing as critical for initial binding to the endothelial cell surface and the β-ladder as essential for downstream NS1-mediated events including cathepsin L activation, both crucial steps for NS1-triggered pathology.

In summary, the structural and mechanistic investigations of 2B7-mediated protection reveal the critical and distinct roles of the NS1 wing domain in cell binding and the β-ladder in downstream signaling. This, coupled with the flavivirus cross-reactivity of 2B7, possessing no risk of ADE, highlights the possibility of treating multiple flavivirus infections with one therapeutic targeting flavivirus NS1.

2B7 Sequences

| | | |
|---|---|---|
| SEQ ID NO: 1 | NCFMH | 2B7 heavy chain |
| | | CDR1 amino acid |
| SEQ ID NO: 2 | YINPYNDMTKYSENFKG | 2B7 heavy chain |
| | | CDR2 amino acid |
| SEQ ID NO: 3 | GYLLRTGCFDY | 2B7 heavy chain |
| | | CDR3 amino acid |
| SEQ ID NO: 4 | RASESVDSYGYSFMH | 2B7 light chain |
| | | CDR1 amino acid |
| SEQ ID NO: 5 | LASNLES | 2B7 light chain |
| | | CDR2 amino acid |
| SEQ ID NO: 6 | QQNNENPLT | 2B7 light chain |
| | | CDR3 amino acid |
| SEQ ID NO: 7 | EVQLQQSGPELVKPGASVKMSCKASGCTLTNCFMH | 2B7 heavy chain |
| | WMKQKPGQDLEWIGYINPYNDMTKYSENFKGKAT | variable region |
| | LTSDKSSSTAFMELSSLTSEDSAVYYCARGYLLRTG | amino acid |
| | CFDYWGQGTTLTVSS | |
| SEQ ID NO: 8 | NIVLTQSPASLAVSLGQRATISCRASESVDSYGYSF | 2B7 light chain |
| | MHWYQQKPGQPPKVLIYLASNLESGVPARFSGSGS | variable region |
| | RTDFTLTIDPVEADDAATYYCQQNNENPLTFGAGT | amino acid |
| | KLELK | |
| SEQ ID NO: 9 | MEWSWIFLFLLSGTAGVHSEVQLQQSGPELVKPGA | 2B7 heavy chain |
| | SVKMSCKASGCTLTNCFMHWMKQKPGQDLEWIGY | amino acid |
| | INPYNDMTKYSENFKGKATLTSDKSSSTAFMELSSL | |
| | TSEDSAVYYCARGYLLRTGCFDYWGQGTTLTVSSA | |
| | KTTPPSVYPLAPGCGDTTGSSVTLGCLVKGYFPESV | |
| | TVTWNSGSLSSSVHTFPALLQSGLYTMSSSVTVPSS | |
| | TWPSQTVTCSVAHPASSTTVDKKLEPSGPISTINPCP | |
| | PCKECHKCPAPNLEGGPSVFIFPPNIKDVLMISLTPK | |
| | VTCVVVDVSEDDPDVQISWFVNNVEVHTAQTQTH | |
| | REDYNSTIRVVSTLPIQHQDWMSGKEFKCKVNNKD | |
| | LPSPIERTISKIKGLVRAPQVYILPPPAEQLSRKDVSL | |
| | TCLVVGFNPGDISVEWTSNGHTEENYKDTAPVLDS | |
| | DGSYFIYSKLNMKTSKWEKTDSFSCNVRHEGLKNY | |
| | YLKKTISRSPGK | |
| SEQ ID NO: 10 | METDTLLLWVLLLWVPGSTGNIVLTQSPASLAVSL | 2B7 Light chain |
| | GQRATISCRASESVDSYGYSFMHWYQQKPGQPPKV | amino acid |
| | LIYLASNLESGVPARFSGSGSRTDFTLTIDPVEADDA | |
| | ATYYCQQNNENPLTFGAGTKLELKRADAAPTVSIFP | |
| | PSSEQLTSGGASVVCFLNNFYPKDINVKWKIDGSER | |
| | QNGVLNSWTDQDSKDSTYSMSSTLTLTKDEYERHN | |
| | SYTCEATHKTSTSPIVKSFNRNEC | |
| SEQ ID NO: 11 | ATGGAATGGAGTTGGATATTTCTCTTTCTCCTGTC | 2B7 heavy chain |
| | AGGAACTGCAGGTGTCCACTCTGAGGTCCAGCTG | DNA sequence |
| | CAGCAGTCTGGACCTGAGCTGGTAAAGCCCGGGG | |
| | CTTCAGTGAAGATGTCCTGCAAGGCTTCTGGATG | |
| | CACACTCACTAACTGTTTTATGCACTGGATGAAG | |
| | CAGAAGCCTGGACAGGACCTTGAGTGGATTGGAT | |
| | ATATTAATCCTTACAATGATATGACTAAGTACAG | |
| | TGAGAACTTCAAAGGCAAGGCCACACTGACTTCA | |
| | GACAAATCCTCCAGCACAGCCTTCATGGAGCTCA | |
| | GCAGCCTGACCTCTGAGGACTCTGCGGTCTATTA | |
| | CTGTGCAAGGGGATATTTACTACGTACGGGCTGC | |
| | TTTGACTACTGGGGCCAAGGCACCACTCTCACAG | |
| | TCTCCTCAGCCAAAACAACACCCCCATCAGTCTA | |
| | TCCACTGGCCCCTGGGTGTGGAGATACAACTGGT | |
| | TCCTCCGTGACTCTGGGATGCCTGGTCAAGGGCT | |
| | ACTTCCCTGAGTCAGTGACTGTGACTTGGAACTCT | |
| | GGATCCCTGTCCAGCAGTGTGCACACCTTCCCAG | |
| | CTCTCCTGCAGTCTGGACTCTACACTATGAGCAG | |
| | CTCAGTGACTGTCCCCTCCAGCACCTGGCCAAGT | |
| | CAGACCGTCACCTGCAGCGTTGCTCACCCAGCCA | |
| | GCAGCACCACGGTGGACAAAAAACTTGAGCCCA | |
| | GCGGGCCCATTTCAACAATCAACCCCTGTCCTCC | |
| | ATGCAAGGAGTGTCACAAATGCCCAGCTCCTAAC | |
| | CTCGAGGGTGGACCATCCGTCTTCATCTTCCCTCC | |
| | AAATATCAAGGATGTACTCATGATCTCCCTGACA | |
| | CCCAAGGTCACGTGTGTGGTGGTGGATGTGAGCG | |
| | AGGATGACCCAGACGTCCAGATCAGCTGGTTTGT | |
| | GAACAACGTGGAAGTACACACAGCTCAGACACA | |
| | AACCCATAGAGAGGATTACAACAGTACTATCCGG | |
| | GTGGTCAGCACCCTCCCCATCCAGCACCAGGACT | |
| | GGATGAGTGGCAAGGAGTTCAAATGCAAGGTCA | |
| | ACAACAAAGACCTCCCATCACCCATCGAGAGAAC | |
| | CATCTCAAAAATTAAAGGGCTAGTCAGAGCTCCA | |
| | CAAGTATACATCTTGCCGCCACCAGCAGAGCAGT | |
| | TGTCCAGGAAAGATGTCAGTCTCACTTGCCTGGT | |
| | CGTGGGCTTCAACCCTGGAGACATCAGTGTGGAG | |
| | TGGACCAGCAATGGGCATACAGAGGAGAACTAC | |
| | AAGGACACCGCACCAGTCCTGGACTCTGACGGTT | |
| | CTTACTTCATATATAGCAAGCTCAATATGAAAAC | |
| | AAGCAAGTGGGAGAAAACAGATTCCTTCTCATGC | |
| | AACGTGAGACACGAGGGTCTGAAAAATTACTACC | |
| | TGAAGAAGACCATCTCCCGGTCTCCGGGTAAATG | |
| | A | |
| SEQ ID NO: 12 | ATGGAGACAGACACACTCCTGCTATGGGTGCTGC | 2B7 light chain |
| | TGCTCTGGGTTCCAGGTTCCACAGGTAACATTGT | DNA sequence |
| | GCTGACCCAATCTCCTGCTTCTTTGGCTGTGTCTC | |
| | TAGGGCAGAGGGCCACCATATCCTGCAGAGCCAG | |
| | TGAAAGTGTTGATAGTTATGGCTATAGTTTTATGC | |
| | ACTGGTACCAGCAGAAACCAGGACAGCCACCCA | |
| | AAGTCCTCATCTATCTTGCATCCAACCTAGAATCT | |
| | GGTGTCCCTGCCAGGTTCAGTGGCAGTGGGTCTA | |
| | GGACAGATTTCACCCTCACCATTGATCCTGTGGA | |
| | GGCTGATGATGCTGCAACTTATTACTGTCAGCAA | |
| | AATAATGAGAATCCGCTCACGTTCGGTGCTGGGA | |
| | CCAAGCTGGAGCTGAAACGGGCTGATGCTGCACC | |
| | AACTGTATCCATCTTCCCACCATCCAGTGAGCAG | |
| | TTAACATCTGGAGGTGCCTCAGTCGTGTGCTTCTT | |
| | GAACAACTTCTACCCCAAAGACATCAATGTCAAG | |
| | TGGAAGATTGATGGCAGTGAACGACAAAATGGC | |
| | GTCCTGAACAGTTGGACTGATCAGGACAGCAAAG | |
| | ACAGCACCTACAGCATGAGCAGCACCCTCACGTT | |
| | GACCAAGGACGAGTATGAACGACATAACAGCTA | |
| | TACCTGTGAGGCCACTCACAAGACATCAACTTCA | |
| | CCCATTGTCAAGAGCTTCAACAGGAATGAGTGTT | |
| | AG | |

Primer Sequences

| Backbone | Mutation | Direction | Sequence | SEQ ID NO: |
|---|---|---|---|---|
| DENV 2 | W115/8A/ | Forward | CTGAGCTGAAGTATTCAGCGAAAACAGCGGCCAAAGCAAAAATGCTCT | 13 |
| | G9A | Reverse | GAGAGCATTTTTGCTTTGGCCGCTGTTTTCGCTGAATACTTCAGCTCAG | 14 |
| DENV 2 | D281P | Forward | GGACTTTGATTTCTGTCCAGGAACAACAGTGGTAGTG | 15 |
| | | Reverse | CACTACCACTGTTGTTCCTGGACAGAAATCAAAGTCC | 16 |
| DENV 2 | R299E | Forward | GAGGACCCTCTTTGGAAACAACCACTGCCTC | 17 |
| | | Reverse | GAGGCAGTGGTTGTTTCCAAAGAGGGTCCTC | 18 |
| DENV 2 | R299A | Forward | GAGGACCCTCTTTGGCAACAACCACTGCC | 19 |
| | | Reverse | GGCAGTGGTTGTTGCCAAAGAGGGTCCTC | 20 |
| DENV 2 | T301K | Forward | GAGGACCCTCTTTGAGAACAAAGACTGCCTCTGG | 21 |
| | | Reverse | CCAGAGGCAGTCTTTGTTCTCAAAGAGGGTCCTC | 22 |
| DENV 2 | T301R | Forward | GAGGACCCTCTTTGAGAACAAGAACTGCCTCTGG | 23 |
| | | Reverse | CCAGAGGCAGTTCTTGTTCTCAAAGAGGGTCCTC | 24 |
| DENV 2 | A303W | Forward | TTGAGAACAACCACTTGGTCTGGAAAACTCATA | 25 |
| | | Reverse | TATGAGTTTTCCAGACCAAGTGGTTGTTCTCAA | 26 |
| DENV 2 | A303R | Forward | TTGAGAACAACCACTCGTTCTGGAAAACTCATA | 27 |
| | | Reverse | TATGAGTTTTCCAGAACGAGTGGTTGTTCTCAA | 28 |
| DENV 2 | G305W | Forward | CCACTGCCTCTTGGAAACTCATAACAGAATGGTGC | 29 |
| | | Reverse | GCACCATTCTGTTATGAGTTTCCAAGAGGCAGTGG | 30 |
| DENV 2 | G305K | Forward | CCACTGCCTCTAAGAAACTCATAACAGAATGGTGC | 31 |
| | | Reverse | GCACCATTCTGTTATGAGTTTCTTAGAGGCAGTGG | 32 |
| DENV 2 | E326K | Forward | GCTAAGATACAGAGGTAAGGATGGGTGCTGGTAC | 33 |
| | | Reverse | GTACCAGCACCCATCCTTACCTCTGTATCTTAGC | 34 |
| DENV 2 | E326A | Forward | CTAAGATACAGAGGTGCAGATGGGTGCTGGTAC | 35 |
| | | Reverse | GTACCAGCACCCATCTGCACCTCTGTATCTTAG | 36 |
| DENV 2 | D327K | Forward | CTAAGATACAGAGGTGAGAAGGGGTGCTGGTACGGGATG | 37 |
| | | Reverse | CATCCCGTACCAGCACCCCTTCTCACCTCTGTATCTTAG | 38 |
| DENV 2 | D327A | Forward | GATACAGAGGTGAGGCTGGGTGCTGGTACG | 39 |
| | | Reverse | CGTACCAGCACCCAGCCTCACCTCTGTATC | 40 |
| DENV 2 | E326K | Forward | CTAAGATACAGAGGTAAGAAGGGGTGCTGGTACGGGATGG | 41 |
| | D327K | Reverse | CCATCCCGTACCAGCACCCCTTCTTACCTCTGTATCTTAG | 42 |
| DENV 2 | E326A | Forward | GATACAGAGGTGCAGCTGGGTGCTGGTACG | 43 |
| | D327A | Reverse | CGTACCAGCACCCAGCTGCACCTCTGTATC | 44 |
| DENV 2 | G328W | Forward | CAGAGGTGAGGATTGGTGCTGGTACGG | 45 |
| | | Reverse | CCGTACCAGCACCAATCCTCACCTCTG | 46 |
| DENV 2 | G328K | Forward | CAGAGGTGAGGATAAGTGCTGGTACGG | 47 |
| | | Reverse | CCGTACCAGCACTTATCCTCACCTCTG | 48 |
| DENV 2 | W330A | Forward | GGTGAGGATGGGTGCGCATACGGGATGG | 49 |
| | | Reverse | CCATCCCGTATGCGCACCCATCCTCACC | 50 |
| DENV 2 | V346E | Forward | GAGAAAGAAGAGAATTTGGAGAACTCCTTGGTCACAGCTC | 51 |
| | | Reverse | GAGCTGTGACCAAGGAGTTCTCCAAATTCTCTTCTTTCTC | 52 |
| DENV 2 | V346F | Forward | GAGAAAGAAGAGAATTTGTTCAACTCCTTGGTCACAGC | 53 |
| | | Reverse | GCTGTGACCAAGGAGTTGAACAAATTCTCTTCTTTCTC | 54 |
| WNV | T301K | Forward | GCCACTCGCACCAAGACAGAGAGCGGA | 55 |
| | | Reverse | TCCGCTCTCTGTCTTGGTGCGAGTGGC | 56 |
| WNV | T301R | Forward | GCCACTCGCACCAGAACAGAGAGCGGA | 57 |
| | | Reverse | TCCGCTCTCTGTTCTGGTGCGAGTGGC | 58 |
| WNV | G305K | Forward | CCACCACAGAGAGCAAAAAGTTGATAACAGATTGG | 59 |
| | | Reverse | CCAATCTGTTATCAACTTTTTGCTCTCTGTGGTGG | 60 |
| ZIKV | T301K | Forward | CCATCTCTGAGATCAAAAACTGCAAGCGGAAGG | 61 |
| | | Reverse | CCTTCCGCTTGCAGTTTTTGATCTCAGAGATGG | 62 |
| ZIKV | T301R | Forward | CCATCTCTGAGATCAAGAACTGCAAGCGGAAGG | 63 |
| | | Reverse | CCTTCCGCTTGCAGTTCTTGATCTCAGAGATGG | 64 |
| ZIKV | G305K | Forward | CCACTGCAAGCAAAAGGGTGATCGAGGAATGG | 65 |
| | | Reverse | CCATTCCTCGATCACCCTTTTGCTTGCAGTGG | 66 |

### REFERENCES

All references, including publications, patent applications, and patents, cited herein are hereby incorporated by reference to the same extent as if each reference were individually and specifically indicated to be incorporated by reference and were set forth in its entirety herein.
1. S. Bhatt et al., Nature 496, 504-507 (2013).
2. S. B. Halstead, Lancet 370, 1644-1652 (2007).
3. P. R. Beatty et al., Sci Transl Med 7, 304ra141 (2015).
4. N. Modhiran et al., Sci Transl Med 7, 304ra142 (2015).
5. A. L. Rothman, Nat Rev Immunol 11, 532-543 (2011).
6. S. Sridhar et al., N Engl J Med 379, 327-340 (2018).
7. L. C. Katzelnick et al., Science 358, 929-932 (2017).
8. P. Scaturro, M. Cortese, L. Chatel-Chaix, W. Fischl, R. Bartenschlager, PLoS Pathog 11, e1005277 (2015).
9. I. Gutsche et al., Proc Natl Acad Sci USA 108, 8003-8008 (2011).
10. S. A. Paranavitane et al., BMC Infect Dis 14, 570 (2014).
11. P. Avirutnan et al., J Exp Med 207, 793-806 (2010).
12. C. H. Chao et al., PLoS Pathog 15, e1007625 (2019).
13. H. Puerta-Guardo, D. R. Glasner, E. Harris, PLoS Pathog 12, e1005738 (2016).
14. D. R. Glasner, H. Puerta-Guardo, P. R. Beatty, E. Harris, Annu Rev Virol, (2018).
15. S. Singh, M. G. Anupriya, A. Modak, E. Sreekumar, J Gen Virol 99, 1658-1670 (2018).
16. H. Puerta-Guardo et al., Cell Rep 26, 1598-1613.e1598 (2019).
17. H. Puerta-Guardo et al., J Infect Dis, (2019).
18. D. A. Espinosa et al., J Immunol 202, 1153-1162 (2019).
19. A. C. Brault et al., Sci Rep 7, 14769 (2017).
20. J. J. Schlesinger, M. W. Brandriss, E. E. Walsh, J Gen Virol 68 (Pt 3), 853-857 (1987).
21. D. L. Akey et al., Science 343, 881-885 (2014).
22. X. Xu et al., EMBO J 35, 2170-2178 (2016).
23. M. A. Edeling, M. S. Diamond, D. H. Fremont, Proc Natl Acad Sci USA 111, 4285-4290 (2014).
24. T. Poonsiri et al., J Virol 92, (2018).
25. H. Song, J. Qi, J. Haywood, Y. Shi, G. F. Gao, Nat Struct Mol Biol 23, 456-458 (2016).
26. D. R. Glasner et al., PLoS Pathog 13, e1006673 (2017).
27. G. J. Xu et al., Viral immunology. Science 348, aaa0698 (2015).
28. Y. C. Chuang, J. Lin, Y. S. Lin, S. Wang, T. M. Yeh, J Immunol 196, 1218-1226 (2016).
29. C. F. Lin et al., J Med Virol 69, 82-90 (2003).
30. W. C. Brown et al., Nat Struct Mol Biol 23, 865-867 (2016).
31. T. Hertz et al., J Immunol 198, 4025-4035 (2017).
32. Y. C. Lai et al., Sci Rep 7, 6975 (2017).

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A binding agent that specifically binds to a flavivirus NS1 protein, which comprises three complementarity determining regions (CDRs) of an antibody heavy chain variable region (VH) and three CDRs of an antibody light chain variable region (LH), wherein:
(a) CDR1 of the VH (HCDR1) comprises the amino acid sequence of SEQ ID NO: 1, CDR2 of the VH (HCDR2) comprises the amino acid sequence of SEQ ID NO: 2, and CDR3 of the VH (HCDR3) comprises the amino acid sequence of SEQ ID NO: 3; and
(b) CDR1 of the LH (LCDR1) comprises the amino acid sequence of SEQ ID NO: 4, CDR2 of the LH (LCDR2) comprises the amino acid sequence of SEQ ID NO: 5, and CDR3 of the LH (LCDR3) comprises the amino acid sequence of SEQ ID NO: 6.

2. The binding agent of claim 1, which comprises an antibody heavy chain variable region and an antibody light chain variable region, wherein the heavy chain variable region comprises the amino acid sequence of SEQ ID NO: 7 or an amino acid sequence that is at least 95% identical to SEQ ID NO: 7 and the light chain variable region comprises the amino acid sequence of SEQ ID NO: 8 or an amino acid sequence that is at least 95% identical to SEQ ID NO: 8.

3. The binding agent of claim 1 or claim 2, which is an antibody or an antigen-binding fragment thereof.

4. The binding agent of claim 3, which is a monoclonal antibody or an antigen-binding fragment thereof.

5. The binding agent of claim 3 or claim 4, which is a chimeric antibody, a humanized antibody, or a human antibody.

6. The binding agent of any one of claims 3-5, which is a whole antibody.

7. The binding agent of any one of claims 3-5, which is an antigen-binding antibody fragment.

8. The binding agent of claim 7, wherein the antibody fragment is selected from F(ab′)₂, Fab′, Fab, Fv, single chain variable fragment (scFv), a disulfide-stabilized Fv fragment (dsFv), a domain antibody (dAb), and a single chain binding polypeptide.

9. The binding agent of claim 8, wherein the antibody fragment is a single chain variable fragment (scFv).

10. The binding agent of claim 1 or claim 2, which is an alternative protein scaffold.

11. The binding agent of any one of claims 1-10, wherein the flavivirus is yellow fever virus, dengue virus, Japanese encephalitis virus, West Nile virus, Zika virus, or St. Louis encephalitis virus.

12. The binding agent of claim 11, wherein the flavivirus is a dengue virus of serotype 1, serotype 2, serotype 3 or serotype 4.

13. A binding agent that specifically binds to a region of a flavivirus NS1 protein comprising one or more of the following amino acid residues:
(a) H269, L270, E281, G282, R299, T301, V303, T304, G305, T307, E326, D327, G328, W330, E343, N344, L345, V346, K347, 5348, and/or M349 of Dengue virus 1 (NCBI Accession No. P17763.2);
(b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990);
(c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843);
(d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2);
(e) H269, S270, P281, G282, R299, T301, A303, S304, G305, V307, K326, D327, G328, W330, S343, N344, L345, V346, R347, S348, and/or M349 of Zika virus (NCBI Accession No. AZS35340);
(f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2);
(g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4);
(h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395);
(i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135);
(j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1);
(k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492);
(l) D271, Q272, P283, G284, R301, T303, E305, S306, G307, I309, G328, T329, D330, W332, G344, G345, L346, V347, R348, S349, and/or M350 of Powassan virus (NCBI Accession No. ACD88752); and/or
(m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

14. The binding agent of claim 13, which comprises three complementarity determining regions (CDRs) of an antibody heavy chain variable region (VH) and three CDRs of an antibody light chain variable region (LH), wherein:
(a) CDR1 of the VH (HCDR1) comprises the amino acid sequence of SEQ ID NO: 1, CDR2 of the VH (HCDR2) comprises the amino acid sequence of SEQ ID NO: 2, and CDR3 of the VH (HCDR3) comprises the amino acid sequence of SEQ ID NO: 3; and
(b) CDR1 of the LH (LCDR1) comprises the amino acid sequence of SEQ ID NO: 4, CDR2 of the LH (LCDR2) comprises the amino acid sequence of SEQ ID NO: 5, and CDR3 of the LH (LCDR3) comprises the amino acid sequence of SEQ ID NO: 6.

15. The binding agent of claim 13 or claim 14, which comprises an antibody heavy chain variable region and an antibody light chain variable region, wherein the heavy chain variable region comprises the amino acid sequence of SEQ ID NO; 7 or an amino acid sequence that is at least 95% identical to SEQ ID NO: 7 and the light chain variable region comprises the amino acid sequence of SEQ ID NO: 8 or an amino acid sequence that is at least 95% identical to SEQ ID NO: 8.

16. The binding agent of claim 1 or claim 2, which is an antibody or an antigen-binding fragment thereof.

17. The binding agent of claim 16, which is an antigen-binding fragment of an antibody.

18. The binding agent of claim 17, which is a single chain variable fragment (scFv).

19. The binding agent of any one of claims 13-15, which is an alternative protein scaffold.

20. The binding agent of any one of claims 13-19, wherein the flavivirus is yellow fever virus, dengue virus, Japanese encephalitis virus, West Nile virus, Zika virus, St. Louis encephalitis virus, tick-borne encephalitis virus, Usutu virus, Powassan virus, or Wesselsbron virus.

21. A conjugate comprising the binding agent of any one of claims 1-20 linked to a therapeutic.

22. The conjugate of claim 21, wherein the therapeutic is an antiviral agent.

23. A composition comprising the binding agent of any one of claims 1-20 or the conjugate of claim 21 or claim 22 and a pharmaceutically acceptable carrier.

24. A method of inducing an immune response against a flavivirus in a mammal, which comprises administering to the mammal the composition of claim 23, whereupon an immune response against the flavivirus is induced in the mammal.

25. The method of claim 24, wherein flavivirus is yellow fever virus, dengue virus, Japanese encephalitis virus, West Nile virus, Zika virus, or St. Louis encephalitis virus.

26. The method of claim 24 or claim 25, wherein the flavivirus is a dengue virus of serotype 1, serotype 2, serotype 3 or serotype 4.

27. Use of a binding agent according to any one of claims 1-20, a conjugate according to claim 21 or claim 22, or a composition according to claim 23 in a method of inducing an immune response against a flavivirus in a mammal.

28. The use of claim 27, wherein flavivirus is yellow fever virus, dengue virus, Japanese encephalitis virus, West Nile virus, Zika virus, or St. Louis encephalitis virus.

29. The use of claim 27 or claim 28, wherein the flavivirus is a dengue virus of serotype 1, serotype 2, serotype 3 or serotype 4.

30. A composition comprising a recombinant flavivirus NS1 antigen and a pharmaceutically acceptable carrier, which recombinant flavivirus NS1 antigen comprises one or more of the following amino acid residues:
(a) H269, L270, E281, G282, R299, T301, V303, T304, G305, T307, E326, D327, G328, W330, E343, N344, L345, V346, K347, 5348, and/or M349 of Dengue virus 1 (NCBI Accession No. P17763.2),
(b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990);
(c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843);
(d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2);
(e) H269, S270, P281, G282, R299, T301, A303, S304, G305, V307, K326, D327, G328, W330, S343, N344, L345, V346, R347, S348, and/or M349 of Zika virus (NCBI Accession No. AZS35340);
(f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2);
(g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4);
(h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395);
(i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135);
(j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1);
(k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492);
(l) D271, Q272, P283, G284, R301, T303, E305, S306, G307, I309, G328, T329, D330, W332, G344, G345, L346, V347, R348, S349, and/or M350 of Powassan virus (NCBI Accession No. ACD88752); or
(m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

31. A composition comprising a nucleic acid sequence encoding a recombinant flavivirus NS1 antigen and pharmaceutically acceptable carrier, wherein the recombinant flavivirus NS1 antigen comprises one or more of the following amino acid residues:
(a) H269, L270, E281, G282, R299, T301, V303, T304, G305, T307, E326, D327, G328, W330, E343, N344, L345, V346, K347, S348, and/or M349 of Dengue virus 1 (NCBI Accession No. P17763.2);
(b) H269, L270, D281, G282, R299, T301, A303, S304, G305, L307, E326, D327, G328, W330, E343, N344, L345, V346, N347, S348, and/or L349 of Dengue virus 2 (NCBI Accession No. P29990);
(c) H269, L270, E281, G282, R299, T301, V303, S304, G305, L307, E326, D327, G328, W330, E343, N344, M345, V346, K347, S348, and/or L349 of Dengue virus 3 (NCBI Accession No. YP_001621843);
(d) H269, L270, P281, G282, R299, T301, A303, S304, G305, L307, E326, D327, G328, W330, E343, N344, M345, V346, K347, S348, and/or Q349 of Dengue virus 4 (NCBI Accession No. P09866.2);
(e) H269, S270, P281, G282, R299, T301, A303, S304, G305, V307, K326, D327, G328, W330, S343, N344, L345, V346, R347, S348, and/or M349 of Zika virus (NCBI Accession No. AZS35340);
(f) S269, E270, P281, G282, R299, T301, A303, S304, G305, L307, K326, D327, G328, W330, A343, K344, L345, V346, K347, S348, and/or R349 of St. Louis encephalitis virus (NCBI Accession No. P09732.2);
(g) D269, E270, P281, G282, R299, T301, E303, S304, G305, L307, D326, S327, G328, W330, K343, T344, L345, V346, Q347, S348, and/or G349 of West Nile virus (NCBI Accession No. Q9Q6P4);
(h) D269, E270, P281, G282, R299, T301, D303, S304, G305, L307, E326, N327, G328, W330, T343, T344, L345, V346, R347, S348, and/or Q349 of Japanese encephalitis virus (NCBI Accession No. P27395);
(i) K270, Y271, P282, G283, R300, T302, E304, S305, G306, V307, G327, T328, D329, W331, G343, G344, L345, V346, R347, S348, and/or M349 of Tick-borne encephalitis virus (NCBI Accession No. NP_043135);
(j) M269, Q270, P281, G282, R299, T301, D303, S304, G305, V307, S326, D327, G328, W330, S343, H344, L345, V346, R347, S348, and/or W349 of Yellow fever virus (NCBI Accession No. NP_041726.1);
(k) D269, E270, P281, G282, R299, T301, S303, S304, G305, L307, K326, N327, G328, W330, T343, T344, L345, V346, K347, S348, and/or S349 of Usutu virus (NCBI Accession No. AWC68492);
(l) D271, Q272, P283, G284, R301, T303, E305, S306, G307, I309, G328, T329, D330, W332, G344, G345, L346, V347, R348, S349, and/or M350 of Powassan virus (NCBI Accession No. ACD88752); or
(m) H270, N271, P282, G283, R300, T302, D304, S305, G306, I308, P327, D328, G329, W331, E343, A344, H345, L346, V347, K348, and/or S349 of Wesselsbron virus (NCBI Accession No. ABI54474).

32. The composition of claim 31, wherein the nucleic acid sequence is present in a vector.

33. A method of inducing an immune response against a flavivirus in a mammal, which comprises administering to the mammal the composition of any one of claims 30-32, whereupon an immune response against the flavivirus is induced in the mammal.


SEQUENCE LISTING (79 sequences)

SEQ ID NO: 1 - protein, 5 aa, Artificial sequence
  Synthetic
NCFMH

SEQ ID NO: 2 - protein, 17 aa, Artificial sequence
  Synthetic
YINPYNDMTKYSENFKG

SEQ ID NO: 3 - protein, 11 aa, Artificial sequence
  Synthetic
GYLLRTGCFDY

SEQ ID NO: 4 - protein, 15 aa, Artificial sequence
  Synthetic
RASESVDSYGYSFMH

SEQ ID NO: 5 - protein, 7 aa, Artificial sequence
  Synthetic
LASNLES

SEQ ID NO: 6 - protein, 9 aa, Artificial sequence
  Synthetic
QQNNENPLT

SEQ ID NO: 7 - protein, 120 aa, Artificial sequence
  Synthetic
EVQLQQSGPELVKPGASVKMSCKASGCTLTNCFMHWMKQKPGQDLEWIGYINPYNDMTKYSENFKGKATLTSDKSSSTAFMELSSLTSEDSAVYYCARGYLLRTGCFDYWGQGTTLTVSS

SEQ ID NO: 8 - protein, 111 aa, Artificial sequence
  Synthetic
NIVLTQSPASLAVSLGQRATISCRASESVDSYGYSFMHWYQQKPGQPPKVLIYLASNLESGVPARFSGSGSRTDFTLTIDPVEADDAATYYCQQNNENPLTFGAGTKLELK

SEQ ID NO: 9 - protein, 475 aa, Artificial sequence
  Synthetic
MEWSWIFLFLLSGTAGVHSEVQLQQSGPELVKPGASVKMSCKASGCTLTNCFMHWMKQKPGQDLEWIGYINPYNDMTKYSENFKGKATLTSDKSSSTAFMELSSLTSEDSAVYYCARGYLLRTGCFDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 10 - protein, 238 aa, Artificial sequence
  Synthetic
METDTLLLWVLLLWVPGSTGNIVLTQSPASLAVSLGQRATISCRASESVDSYGYSFMHWYQQKPGQPPKVLIYLASNLESGVPARFSGSGSRTDFTLTIDPVEADDAATYYCQQNNENPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - DNA, 1428 bp, Artificial sequence
  Synthetic
atggaatggagttggatatttctctttctcctgtcaggaactgcaggtgtccactctgaggtccagctgcagcagtctggacctgagctggtaaagcccggggcttcagtgaagatgtcctgcaaggcttctggatgcacactcactaactgttttatgcactggatgaagcagaagcctggacaggaccttgagtggattggatatattaatccttacaatgatatgactaagtacagtgagaacttcaaaggcaaggccacactgacttcagacaaatcctccagcacagccttcatggagctcagcagcctgacctctgaggactctgcggtctattactgtgcaaggggatatttactacgtacgggctgctttgactactggggccaaggcaccactctcacagtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 12 - DNA, 717 bp, Artificial sequence
  Synthetic
atggagacagacacactcctgctatgggtgctgctgctctgggttccaggttccacaggtaacattgtgctgacccaatctcctgcttctttggctgtgtctctagggcagagggccaccatatcctgcagagccagtgaaagtgttgatagttatggctatagttttatgcactggtaccagcagaaaccaggacagccacccaaagtcctcatctatcttgcatccaacctagaatctggtgtccctgccaggttcagtggcagtgggtctaggacagatttcaccctcaccattgatcctgtggaggctgatgatgctgcaacttattactgtcagcaaaataatgagaatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 13 - DNA, 48 bp, Artificial sequence
  Synthetic
ctgagctgaagtattcagcgaaaacagcggccaaagcaaaaatgctct

SEQ ID NO: 14 - DNA, 49 bp, Artificial sequence
  Synthetic
gagagcatttttgctttggccgctgttttcgctgaatacttcagctcag

SEQ ID NO: 15 - DNA, 37 bp, Artificial sequence
  Synthetic
ggactttgatttctgtccaggaacaacagtggtagtg

SEQ ID NO: 16 - DNA, 37 bp, Artificial sequence
  Synthetic
cactaccactgttgttcctggacagaaatcaaagtcc

SEQ ID NO: 17 - DNA, 31 bp, Artificial sequence
  Synthetic
gaggaccctctttggaaacaaccactgcctc

SEQ ID NO: 18 - DNA, 31 bp, Artificial sequence
  Synthetic
gaggcagtggttgtttccaaagagggtcctc

SEQ ID NO: 19 - DNA, 29 bp, Artificial sequence
  Synthetic
gaggaccctctttggcaacaaccactgcc

SEQ ID NO: 20 - DNA, 29 bp, Artificial sequence
  Synthetic
ggcagtggttgttgccaaagagggtcctc

SEQ ID NO: 21 - DNA, 34 bp, Artificial sequence
  Synthetic
gaggaccctctttgagaacaaagactgcctctgg

SEQ ID NO: 22 - DNA, 34 bp, Artificial sequence
  Synthetic
ccagaggcagtctttgttctcaaagagggtcctc

SEQ ID NO: 23 - DNA, 34 bp, Artificial sequence
  Synthetic
gaggaccctctttgagaacaagaactgcctctgg

SEQ ID NO: 24 - DNA, 34 bp, Artificial sequence
  Synthetic
ccagaggcagttcttgttctcaaagagggtcctc

SEQ ID NO: 25 - DNA, 33 bp, Artificial sequence
  Synthetic
ttgagaacaaccacttggtctggaaaactcata

SEQ ID NO: 26 - DNA, 33 bp, Artificial sequence
  Synthetic
tatgagttttccagaccaagtggttgttctcaa

SEQ ID NO: 27 - DNA, 33 bp, Artificial sequence
  Synthetic
ttgagaacaaccactcgttctggaaaactcata

SEQ ID NO: 28 - DNA, 33 bp, Artificial sequence
  Synthetic
tatgagttttccagaacgagtggttgttctcaa

SEQ ID NO: 29 - DNA, 35 bp, Artificial sequence
  Synthetic
ccactgcctcttggaaactcataacagaatggtgc

SEQ ID NO: 30 - DNA, 35 bp, Artificial sequence
  Synthetic
gcaccattctgttatgagtttccaagaggcagtgg

SEQ ID NO: 31 - DNA, 35 bp, Artificial sequence
  Synthetic
ccactgcctctaagaaactcataacagaatggtgc

SEQ ID NO: 32 - DNA, 35 bp, Artificial sequence
  Synthetic
gcaccattctgttatgagtttcttagaggcagtgg

SEQ ID NO: 33 - DNA, 34 bp, Artificial sequence
  Synthetic
gctaagatacagaggtaaggatgggtgctggtac

SEQ ID NO: 34 - DNA, 34 bp, Artificial sequence
  Synthetic
gtaccagcacccatccttacctctgtatcttagc

SEQ ID NO: 35 - DNA, 33 bp, Artificial sequence
  Synthetic
ctaagatacagaggtgcagatgggtgctggtac

SEQ ID NO: 36 - DNA, 33 bp, Artificial sequence
  Synthetic
gtaccagcacccatctgcacctctgtatcttag

SEQ ID NO: 37 - DNA, 39 bp, Artificial sequence
  Synthetic
ctaagatacagaggtgagaaggggtgctggtacgggatg

SEQ ID NO: 38 - DNA, 39 bp, Artificial sequence
  Synthetic
catcccgtaccagcaccccttctcacctctgtatcttag

SEQ ID NO: 39 - DNA, 30 bp, Artificial sequence
  Synthetic
gatacagaggtgaggctgggtgctggtacg

SEQ ID NO: 40 - DNA, 30 bp, Artificial sequence
  Synthetic
cgtaccagcacccagcctcacctctgtatc

SEQ ID NO: 41 - DNA, 40 bp, Artificial sequence
  Synthetic
ctaagatacagaggtaagaaggggtgctggtacgggatgg

SEQ ID NO: 42 - DNA, 40 bp, Artificial sequence
  Synthetic
ccatcccgtaccagcaccccttcttacctctgtatcttag

SEQ ID NO: 43 - DNA, 30 bp, Artificial sequence
  Synthetic
gatacagaggtgcagctgggtgctggtacg

SEQ ID NO: 44 - DNA, 30 bp, Artificial sequence
  Synthetic
cgtaccagcacccagctgcacctctgtatc

SEQ ID NO: 45 - DNA, 27 bp, Artificial sequence
  Synthetic
cagaggtgaggattggtgctggtacgg

SEQ ID NO: 46 - DNA, 27 bp, Artificial sequence
  Synthetic
ccgtaccagcaccaatcctcacctctg

SEQ ID NO: 47 - DNA, 27 bp, Artificial sequence
  Synthetic
cagaggtgaggataagtgctggtacgg

SEQ ID NO: 48 - DNA, 27 bp, Artificial sequence
  Synthetic
ccgtaccagcacttatcctcacctctg

SEQ ID NO: 49 - DNA, 28 bp, Artificial sequence
  Synthetic
ggtgaggatgggtgcgcatacgggatgg

SEQ ID NO: 50 - DNA, 28 bp, Artificial sequence
  Synthetic
ccatcccgtatgcgcacccatcctcacc

SEQ ID NO: 51 - DNA, 40 bp, Artificial sequence
  Synthetic
gagaaagaagagaatttggagaactccttggtcacagctc

SEQ ID NO: 52 - DNA, 40 bp, Artificial sequence
  Synthetic
gagctgtgaccaaggagttctccaaattctcttctttctc

SEQ ID NO: 53 - DNA, 38 bp, Artificial sequence
  Synthetic
gagaaagaagagaatttgttcaactccttggtcacagc

SEQ ID NO: 54 - DNA, 38 bp, Artificial sequence
  Synthetic
gctgtgaccaaggagttgaacaaattctcttctttctc

SEQ ID NO: 55 - DNA, 27 bp, Artificial sequence
  Synthetic
gccactcgcaccaagacagagagcgga

SEQ ID NO: 56 - DNA, 27 bp, Artificial sequence
  Synthetic
tccgctctctgtcttggtgcgagtggc

SEQ ID NO: 57 - DNA, 27 bp, Artificial sequence
  Synthetic
gccactcgcaccagaacagagagcgga

SEQ ID NO: 58 - DNA, 27 bp, Artificial sequence
  Synthetic
tccgctctctgttctggtgcgagtggc

SEQ ID NO: 59 - DNA, 35 bp, Artificial sequence
  Synthetic
ccaccacagagagcaaaaagttgataacagattgg

SEQ ID NO: 60 - DNA, 35 bp, Artificial sequence
  Synthetic
ccaatctgttatcaactttttgctctctgtggtgg

SEQ ID NO: 61 - DNA, 33 bp, Artificial sequence
  Synthetic
ccatctctgagatcaaaaactgcaagcggaagg

SEQ ID NO: 62 - DNA, 33 bp, Artificial sequence
  Synthetic
ccttccgcttgcagtttttgatctcagagatgg

SEQ ID NO: 63 - DNA, 33 bp, Artificial sequence
  Synthetic
ccatctctgagatcaagaactgcaagcggaagg

SEQ ID NO: 64 - DNA, 33 bp, Artificial sequence
  Synthetic
ccttccgcttgcagttcttgatctcagagatgg

SEQ ID NO: 65 - DNA, 32 bp, Artificial sequence
  Synthetic
ccactgcaagcaaaagggtgatcgaggaatgg

SEQ ID NO: 66 - DNA, 32 bp, Artificial sequence
  Synthetic
ccattcctcgatcacccttttgcttgcagtgg

SEQ ID NO: 67 - protein, 352 aa, Dengue virus serotype 1
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDFCEGTTVVVDEHCGNRGPSLRTTTVTGKIIHEWCCRSCTLPPLRFKGEDGCWYGMEIRPVKDKEENLVKSMVSA

SEQ ID NO: 68 - protein, 352 aa, Dengue virus serotype 2
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTA

SEQ ID NO: 69 - protein, 352 aa, Dengue virus serotype 3
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSA

SEQ ID NO: 70 - protein, 352 aa, Dengue virus serotype 4
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTA

SEQ ID NO: 71 - protein, 352 aa, Zika virus
DVGCSVDFSKKETRCGTGVFIYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLEHRAWNSFLVEDHGFGVFHTSVWLKVREDYSLECDPAVIGTAVKGREAAHSDLGYWIESEKNDTWRLKRAHLIEMKTCEWPKSHTLWTDGVEESDLIIPKSLAGPLSHHNTREGYRTQVKGPWHSEELEIRFEECPGTKVYVEETCGTRGPSLRSTTASGRVIEEWCCRECTMPPLSFRAKDGCWYGMEIRPRKEPESNLVRSMVTA

SEQ ID NO: 72 - protein, 352 aa, Saint Louis encephalitis virus
DSGCAIDLQRRELKCGGGIFVYNDVEKWKSDYKYFPLTPTGLAHVIQEAHANGICGIRSTSRLEHLMWENIQRELNAIFEDNEIDLSVVVQEDPKYYKRAPRRLKKLEDELDYGWKKWGKTLFVEPRLGNNTFVVDGPETKECPTANRAWNSFKVEDFGFGMVFTRLWLTIREENTTECDSAIIGTAIKGDRAVHSDLSYWIESKKNETWQLERAVMGEVKSCTWPETHTLWGDGVVESEMIIPVTLGGPKSHHNKRNGYHTQTKGPWSEGEITLDFDYCPGTTVTVTEHCGNRGASLRTTTASGKLVTDWCCRSCSLPPLRYTTKDGCWYGMEIRPVKEEEAKLVKSRVTA

SEQ ID NO: 73 - protein, 352 aa, West Nile virus
DTGCAIDISRQELRCGSGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNA

SEQ ID NO: 74 - protein, 352 aa, Japanese encephalitis virus
DTGCAIDITRKEMRCGSGIFVHNDVEAWVDRYKYLPETPRSLAKIVHKAHKEGVCGVRSVTRLEHQMWEAVRDELNVLLKENAVDLSVVVNKPVGRYRSAPKRLSMTQEKFEMGWKAWGKSILFAPELANSTFVVDGPETKECPDEHRAWNSMQIEDFGFGITSTRVWLKIREESTDECDGAIIGTAVKGHVAVHSDLSYWIESRYNDTWKLERAVFGEVKSCTWPETHTLWGDDVEESELIIPHTIAGPKSKHNRREGYKTQNQGPWDENGIVLDFDYCPGTKVTITEDCSKRGPSVRTTTDSGKLITDWCCRSCSLPPLRFRTENGCWYGMEIRPVMHDETTLVRSQVDA

SEQ ID NO: 75 - protein, 352 aa, Usutu virus
DSGCAIDVGRRELRCGQGIFIHNDVEAWVDRYKFMPETPKQLAKVIEQAHAKGICGLRSVSRLEHVMWENIRDELNTLLRENAVDLSVVVEKPKGMYKSAPQRLALTSEEFEIGWKAWGKSLVFAPELANHTFVVDGPETKECPDVKRAWNSLEIEDFGFGIMSTRVWLKVREHNTTDCDSSIIGTAVKGDIAVHSDLSYWIESHKNTTWRLERAVFGEIKSCTWPETHTLWSDGVVESDLVVPVTLAGPKSNHNRREGYKVQSQGPWDEEDIVLDFDYCPGTTVTITEACGKRGPSIRTTTSSGRLVTDWCCRSCTLPPLRYKTKNGCWYGMEIRPMKHDETTLVKSSVSA

SEQ ID NO: 76 - protein, 353 aa, Wesselsbron virus
EVGCSLSWKQREMKCGDGVFVFKDSDDWFSKYQYIPEDPKTMATLIHQAHQDGLCGLSSVSDLEHRMWYSRVDEINAILDENEVDLTVVVQESDAVYLRGSHAFPRPKSELKYGWKTWGKNIIFNPSRKNGTFIIDGKSKAECPFNKRVWNSIRVEEFGTGVYQTRVFMRPEFDYTKLCDTGTLGAAVKGSVSAHGDPMFWMESEEINGTWMITTLEALNYRECEWPSSHTLDGAKVVESDMFMPRSLAGPISKHNHIPGYKVQTSGPWHNVPLEIKREECPGTTVVVDEKCDDRAKSVRSTTDSGKIIPEWCCRSCTMPPVSFWGPDGCWYSMEVRPKHTNEAHLVKSWVVA

SEQ ID NO: 77 - protein, 352 aa, Tick-borne encephalitis virus
DVGCAVDTERMELRCGEGLVVWREVSEWYDNYAYYPETPGALASAIKETFEEGSCGVVPQNRLEMAMWRSSVTELNLALAEGEANLTVVVDKFDPTDYRGGVPGLLKKGKDIKVSWKSWGHSMIWSIPEAPRRFMVGTEGQSECPLERRKTGVFTVAEFGVGLRTKVFLDFRQEPTHECDTGVMGAAVKNGMAIHTDQSLWMRSMKNDTGTYIVELLVTDLRNCSWPASHTIDNADVVDSELFLPASLAGPRSWYNRIPGYSEQVKGPWKYTPIRVIREECPGTTVTINAKCDKRGASVRSTTESGKVIPEWCCRACTMPPVTFRTGTDCWYAMEIRPVHDQGGLVRSMVVA

SEQ ID NO: 78 - protein, 353 aa, Powassan virus
DYGCAIDPERMEIRCGEGLVVWKEVSEWYDGYAYHPESPDTLAQALREAFERGVCGVVPQNRLEMAMWRSTAPELNLVLSEGEANLTIVVDKTDPADYRGGTPMVLKKTGKESKVSWKSWGKSILWSVPDSPRRMMMGVDGVGECPLYRRATGVFTVAEFGVGLRTKVFLDLRGEASKECDTGVMGAAVKNGKAIHTDQSMWMSSFRNDTGTYIHELILTDLRNCTWPASHTIDNDGVLDSHLFLPVTLAGPRSKYNRIPGYSEQVRGPWDQTPLRVVRDHCPGTSVRIDSHCDKRGASVRSTTESGKIIPEWCCRACELPPVTFRSGTDCWYAMEIRPVHSQGGLVRSMVVA

SEQ ID NO: 79 - protein, 352 aa, Yellow fever virus
DQGCAINFGKRELKCGDGIFIFRDSDDWLNKYSYYPEDPVKLASIVKASFEEGKCGLNSVDSLEHEMWRSRADEINAIFEENEVDISVVVQDPKNVYQRGTHPFSRIRDGLQYGWKTWGKNLVFSPGRKNGSFIIDGKSRKECPFSNRVWNSFQIEEFGTGVFTTRVYMDAVFEYTIDCDGSILGAAVNGKKSAHGSPTFWMGSHEVNGTWMIHTLEALDYKECEWPLTHTIGTSVEESEMFMPRSIGGPVSSHNHIPGYKVQTNGPWMQVPLEVKREACPGTSVIIDGNCDGRGKSTRSTTDSGKVIPEWCCRSCTMPPVSFHGSDGCWYPMEIRPRKTHESHLVRSWVTA